SEQUENCE LISTING (85 sequences)

SEQ ID NO: 1 - protein, 132 aa, Bacteriophage Q beta
AKLETVTLGNIGKDGKQTLVLNPRGVNPTNGVASLSQAGAVPALEKRVTVSVSQPSRNRKNYKVQVKIQNPTACTANGSCDPSVTRQAYADVTFSFTQYSTDEERAFVRTELAALLASPLLIDAIDQLNPAY

SEQ ID NO: 2 - protein, 329 aa, Bacteriophage Q beta
MAKLETVTLGNIGKDGKQTLVLNPRGVNPTNGVASLSQAGAVPALEKRVTVSVSQPSRNRKNYKVQVKIQNPTACTANGSCDPSVTRQAYADVTFSFTQYSTDEERAFVRTELAALLASPLLIDAIDQLNPAYWTLLIAGGGSGSKPDPVIPDPPIDPPPGTGKYTCPFAIWSLEEVYEPPTKNRPWPIYNAVELQPREFDVALKDLLGNTKWRDWDSRLSYTTFRGCRGNGYIDLDATYLATDQAMRDQKYDIREGKKPGAFGNIERFIYLKSINAYCSLSDIAAYHADGVIVGFWRDPSSGGAIPFDFTKFDKTKCPIQAVIVVPRA

SEQ ID NO: 3 - protein, 130 aa, Bacteriophage fr
MASNFEEFVLVDNGGTGDVKVAPSNFANGVAEWISSNSRSQAYKVTCSVRQSSANNRKYTVKVEVPKVATQVQGGVELPVAAWRSYMNMELTIPVFATNDDCALIVKALQGTFKTGNPIATAIAANSGIY

SEQ ID NO: 4 - protein, 130 aa, Bacteriophage GA
MATLRSFVLVDNGGTGNVTVVPVSNANGVAEWLSNNSRSQAYRVTASYRASGADKRKYAIKLEVPKIVTQVVNGVELPGSAWKAYASIDLTIPIFAATDDVTVISKSLAGLFKVGNPIAEAISSQSGFYA

SEQ ID NO: 5 - protein, 128 aa, Bacteriophage PP7
MSKTIVLSVGEATRTLTEIQSTADRQIFEEKVGPLVGRLRLTASLRQNGAKTAYRVNLKLDQADVVDCSTSVCGELPKVRYTQVWSHDVTIVANSTEASRKSLYDLTKSLVATSQVEDLVVNLVPLGR

SEQ ID NO: 6 - protein, 132 aa, Bacteriophage Q-beta
AKLETVTLGNIGRDGKQTLVLNPRGVNPTNGVASLSQAGAVPALEKRVTVSVSQPSRNRKNYKVQVKIQNPTACTANGSCDPSVTRQKYADVTFSFTQYSTDEERAFVRTELAALLASPLLIDAIDQLNPAY

SEQ ID NO: 7 - protein, 132 aa, Bacteriophage Q-beta
AKLETVTLGKIGKDGKQTLVLNPRGVNPTNGVASLSQAGAVPALEKRVTVSVSQPSRNRKNYKVQVKIQNPTACTANGSCDPSVTRQKYADVTFSFTQYSTDEERAFVRTELAALLASPLLIDAIDQLNPAY

SEQ ID NO: 8 - protein, 132 aa, Bacteriophage Q-beta
ARLETVTLGNIGRDGKQTLVLNPRGVNPTNGVASLSQAGAVPALEKRVTVSVSQPSRNRKNYKVQVKIQNPTACTANGSCDPSVTRQKYADVTFSFTQYSTDEERAFVRTELAALLASPLLIDAIDQLNPAY

SEQ ID NO: 9 - protein, 132 aa, Bacteriophage Q-beta
AKLETVTLGNIGKDGRQTLVLNPRGVNPTNGVASLSQAGAVPALEKRVTVSVSQPSRNRKNYKVQVKIQNPTACTANGSCDPSVTRQKYADVTFSFTQYSTDEERAFVRTELAALLASPLLIDAIDQLNPAY

SEQ ID NO: 10 - protein, 132 aa, Bacteriophage Q-beta
ARLETVTLGNIGKDGRQTLVLNPRGVNPTNGVASLSQAGAVPALEKRVTVSVSQPSRNRKNYKVQVKIQNPTACTANGSCDPSVTRQKYADVTFSFTQYSTDEERAFVRTELAALLASPLLIDAIDQLNPAY

SEQ ID NO: 11 - protein, 185 aa, Hepatitis B virus
MDIDPYKEFGATVELLSFLPSDFFPSVRDLLDTASALYREALESPEHCSPHHTALRQAILCWGELMTLATWVGNNLEDPASRDLVVNYVNTNMGLKIRQLLWFHISCLTFGRETVLEYLVSFGVWIRTPPAYRPPNAPILSTLPETTVVRRRDRGRSPRRRTPSPRRRRSQSPRRRRSQSRESQC

SEQ ID NO: 12 - protein, 212 aa, Hepatitis B virus
MQLFHLCLIISCSCPTVQASKLCLGWLWGMDIDPYKEFGATVELLSFLPSDFFPSVRDLLDTASALYREALESPEHCSPHHTALRQAILCWGDLMNLATWVGGNLEDPVSRDLVVGYVNTTVGLKFRQLLWFHISCLTFGRETVIEYLVSFGVWIRTPPAYRPPNAPILSTLPETTVVRRRGRSPRRRTPSPRRRRSQSPRRRRSQSRESQC

SEQ ID NO: 13 - protein, 188 aa, Hepatitis B virus
MDIDPYKEFGSSYQLLNFLPLDFFPDLNALVDTATALYEEELTGREHCSPHHTAIRQALVCWDELTKLIAWMSSNITSEQVRTIIVNHVNDTWGLKVRQSLWFHLSCLTFGQHTVQEFLVSFGVWIRTPAPYRPPNAPILSTLPEHTVIRRRGGARASRSPRRRTPSPRRRRSQSPRRRRSQSPSTNC

SEQ ID NO: 14 - protein, 185 aa, Artificial Sequence; identical to SEQ ID NO: 11
  Hepatitis B virus variant

SEQ ID NO: 15 - protein, 152 aa, Artificial Sequence
  HBcAg variant
MDIDPYKEFGATVELLSFLPSDFFPSVRDLLDTAAALYRDALESPEHCSPHHTALRQAILCWGDLMTLATWVGTNLEDGGKGGSRDLVVSYVNTNVGLKFRQLLWFHISCLTFGRETVLEYLVSFGVWIRTPPAYRPPNAPILSTLPETTVV

SEQ ID NO: 16 - DNA, 3635 bp, Artificial Sequence
  Plasmid, pAP283-58, encoding RNA phage AP205 coat protein
cgagctcgcccctggcttatcgaaattaatacgactcactatagggagaccggaattcgagctcgcccggggatcctctagaattttctgcgcacccatcccgggtggcgcccaaagtgaggaaaatcacatggcaaataagccaatgcaaccgatcacatctacagcaaataaaattgtgtggtcggatccaactcgtttatcaactacattttcagcaagtctgttacgccaacgtgttaaagttggtatagccgaactgaataatgtttcaggtcaatatgtatctgtttataagcgtcctgcacctaaaccggaaggttgtgcagatgcctgtgtcattatgccgaatgaaaaccaatccattcgcacagtgatttcagggtcagccgaaaacttggctaccttaaaagcagaatgggaaactcacaaacgtaacgttgacacactcttcgcgagcggcaacgccggtttgggtttccttgaccctactgcggctatcgtatcgtctgatactactgcttaagcttgtattctatagtgtcacctaaatcgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgttccgcaccccggaaatggtcaccgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgatatggtgcactctcagtacaatctgctctgatgccgcatagttaagccaactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgcgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggtgtcatggtcggtgatcgccagggtgccgacgcgcatctcgactgcatggtgcaccaatgcttctggcgtcaggcagccatcggaagctgtggtatggccgtgcaggtcgtaaatcactgcataattcgtgtcgctcaaggcgcactcccgttctggataatgttttttgcgccgacatcataacggttctggcaaatattctgaaatgagctgttgacaattaatcatcgaactagttaactagtacgcaagttcacgtaaaaagggtatcgcggaatt

SEQ ID NO: 17 - DNA, 35 bp, Artificial Sequence
  vector pQb185
tctagattaacccaacgcgtaggagtcaggccatg

SEQ ID NO: 18 - protein, 131 aa, Bacteriophage AP205
MANKPMQPITSTANKIVWSDPTRLSTTFSASLLRQRVKVGIAELNNVSGQYVSVYKRPAPKPEGCADACVIMPNENQSIRTVISGSAENLATLKAEWETHKRNVDTLFASGNAGLGFLDPTAAIVSSDTTA

SEQ ID NO: 19 - protein, 131 aa, Bacteriophage AP205
MANKTMQPITSTANKIVWSDPTRLSTTFSASLLRQRVKVGIAELNNVSGQYVSVYKRPAPKPEGCADACVIMPNENQSIRTVISGSAENLATLKAEWETHKRNVDTLFASGNAGLGFLDPTAAIVSSDTTA

SEQ ID NO: 20 - DNA, 3613 bp, Artificial Sequence
  Plasmid, pAP281-32, encoding RNA phage AP205 coat protein
cgagctcgcccctggcttatcgaaattaatacgactcactatagggagaccggaattcgagctcgcccggggatcctctagattaacccaacgcgtaggagtcaggccatggcaaataagacaatgcaaccgatcacatctacagcaaataaaattgtgtggtcggatccaactcgtttatcaactacattttcagcaagtctgttacgccaacgtgttaaagttggtatagccgaactgaataatgtttcaggtcaatatgtatctgtttataagcgtcctgcacctaaaccggaaggttgtgcagatgcctgtgtcattatgccgaatgaaaaccaatccattcgcacagtgatttcagggtcagccgaaaacttggctaccttaaaagcagaatgggaaactcacaaacgtaacgttgacacactcttcgcgagcggcaacgccggtttgggtttccttgaccctactgcggctatcgtatcgtctgatactactgcttaagcttgtattctatagtgtcacctaaatcgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgttccgcaccccggaaatggtcaccgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgatatggtgcactctcagtacaatctgctctgatgccgcatagttaagccaactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgcgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggtgtcatggtcggtgatcgccagggtgccgacgcgcatctcgactgcatggtgcaccaatgcttctggcgtcaggcagccatcggaagctgtggtatggccgtgcaggtcgtaaatcactgcataattcgtgtcgctcaaggcgcactcccgttctggataatgttttttgcgccgacatcataacggttctggcaaatattctgaaatgagctgttgacaattaatcatcgaactagttaactagtacgcaagttcacgtaaaaagggtatcgcggaatt

SEQ ID NO: 21 - protein, 9 aa, Artificial Sequence
  N terminal glycine serine linkers
  REPEAT (1)..(1): Glycine can be repeated from zero to five times
  REPEAT (3)..(3): Glycine can be repeated from zero to ten times
  REPEAT (4)..(4): Serine can be repeated from zero to two times
  REPEAT (5)..(9): These residues can be repeated from zero to three times as a group
GCGSGGGGS

SEQ ID NO: 22 - protein, 10 aa, Artificial Sequence
  C terminal glycine serine linkers
  REPEAT (1)..(1): Glycine can be repeated from zero to ten times
  REPEAT (2)..(2): Serine can be repeated from zero to two times
  REPEAT (3)..(7): These residues can be repeated from zero to three times as a group
  REPEAT (8)..(8): Glycine can be repeated from zero to eight times
  REPEAT (10)..(10): Glycine can be repeated from zero to five times
GSGGGGSGCG

SEQ ID NO: 23 - protein, 5 aa, Artificial Sequence
  Glycine serine linker
GGGGS

SEQ ID NO: 24 - protein, 10 aa, Artificial Sequence
  N-terminal gamma1
CGDKTHTSPP

SEQ ID NO: 25 - protein, 10 aa, Artificial Sequence
  C terminal gamma 1
DKTHTSPPCG

SEQ ID NO: 26 - protein, 17 aa, Artificial Sequence
  N terminal gamma 3
CGGPKPSTPPGSSGGAP

SEQ ID NO: 27 - protein, 18 aa, Artificial Sequence
  C terminal gamma 3
PKPSTPPGSSGGAPGGCG

SEQ ID NO: 28 - protein, 6 aa, Artificial Sequence
  N terminal glycine linker
GCGGGG

SEQ ID NO: 29 - protein, 6 aa, Artificial Sequence
  C terminal glycine linker
GGGGCG

SEQ ID NO: 30 - protein, 6 aa, Artificial Sequence
  C terminal glycine-lysine linker
GGKKGC

SEQ ID NO: 31 - protein, 6 aa, Artificial Sequence
  N terminal glycine lysine linker
CGKKGG

SEQ ID NO: 32 - protein, 4 aa, Artificial Sequence
  C terminal linker
GGCG

SEQ ID NO: 33 - DNA, 31 bp, Artificial Sequence
  oligonucleotide primer
ccggaattcatggacattgacccttataaag

SEQ ID NO: 34 - DNA, 51 bp, Artificial Sequence
  oligonucleotide primer
cctagagccacctttgccaccatcttctaaattagtacccacccaggtagc

SEQ ID NO: 35 - DNA, 48 bp, Artificial Sequence
  oligonucleotide primer
gaagatggtggcaaaggtggctctagggacctagtagtcagttatgtc

SEQ ID NO: 36 - DNA, 38 bp, Artificial Sequence
  oligonucleotide primer
cgcgtcccaagcttctaacattgagattcccgagattg

SEQ ID NO: 37 - protein, 10 aa, Artificial Sequence
  epitope CepsilonH3
VNLTWSRASG

SEQ ID NO: 38 - DNA, 51 bp, Artificial Sequence
  oligonucleotide primer
  CDS (1)..(51)
gttaacttgacctggtctcgtgcttctggtgcatccagggatctagtagtc

SEQ ID NO: 39 - protein, 17 aa, Artificial Sequence
  oligonucleotide primer
VNLTWSRASGASRDLVV

SEQ ID NO: 40 - DNA, 51 bp, Artificial Sequence
  oligonucleotide primer
  CDS (31)..(51)
accagaagcacgagaccaggtcaagttaacatcttccaaattattacccac

SEQ ID NO: 41 - protein, 7 aa, Artificial Sequence
  oligonucleotide primer
IFQIITH

SEQ ID NO: 42 - protein, 9 aa, Homo sapiens
KTWGQYWQV

SEQ ID NO: 43 - protein, 9 aa, Homo sapiens
ITDQVPFSV

SEQ ID NO: 44 - protein, 9 aa, Homo sapiens
YLEPGPVTA

SEQ ID NO: 45 - protein, 10 aa, Homo sapiens
LLDGTATLRL

SEQ ID NO: 46 - protein, 10 aa, Homo sapiens
VLYRYGSFSV

SEQ ID NO: 47 - protein, 9 aa, Homo sapiens
AAGIGILTV

SEQ ID NO: 48 - protein, 9 aa, Homo sapiens
ILTVILGVL

SEQ ID NO: 49 - protein, 9 aa, Homo sapiens
MLLAVLYCL

SEQ ID NO: 50 - protein, 9 aa, Homo sapiens
YMDGTMSQV

SEQ ID NO: 51 - protein, 9 aa, Homo sapiens
VLPDVFIRC

SEQ ID NO: 52 - protein, 9 aa, Homo sapiens
FLWGPRALV

SEQ ID NO: 53 - protein, 9 aa, Homo sapiens
YLSGANLNL

SEQ ID NO: 54 - protein, 9 aa, Homo sapiens
RMPEAAPPV

SEQ ID NO: 55 - protein, 9 aa, Homo sapiens
STPPPGTRV

SEQ ID NO: 56 - protein, 9 aa, Homo sapiens
LLGRNSFEV

SEQ ID NO: 57 - protein, 9 aa, Homo sapiens
KIFGSLAFL

SEQ ID NO: 58 - protein, 9 aa, Homo sapiens
IISAVVGIL

SEQ ID NO: 59 - protein, 8 aa, Homo sapiens
TLGIVCPI

SEQ ID NO: 60 - protein, 5 aa, Artificial Sequence
  HBcAg peptide
GGKGG

SEQ ID NO: 61 - protein, 185 aa, Artificial Sequence
  HBcAg variant
MDIDPYKEFGATVELLSFLPSDFFPSVRDLLDTASALYREALESPEHSSPHHTALRQAILCWGELMTLATWVGNNLEDPASRDLVVNYVNTNMGLKIRQLLWFHISSLTFGRETVLEYLVSFGVWIRTPPAYRPPNAPILSTLPETTVVRRRDRGRSPRRRTPSPRRRRSQSPRRRRSQSRESQC

SEQ ID NO: 62 - protein, 152 aa, Artificial Sequence
  HBcAg
MDIDPYKEFGATVELLSFLPSDFFPSVRDLLDTAAALYRDALESPEHSSPHHTALRQAILCWGDLMTLATWVGTNLEDGGKGGSRDLVVSYVNTNVGLKFRQLLWFHISSLTFGRETVLEYLVSFGVWIRTPPAYRPPNAPILSTLPETTVV

SEQ ID NO: 63 - DNA, 20 bp, Artificial Sequence
  HB1 primer
  misc_feature (3)..(3): k is g or t
  misc_feature (6)..(6): r is a or g
  misc_feature (7)..(7): m is a or c
gakgtrmagcttcaggagtc

SEQ ID NO: 64 - DNA, 20 bp, Artificial Sequence
  HB2 primer
  misc_feature (6)..(6): b is g or c or t
  misc_feature (12)..(12): b is g or c or t
gaggtbcagctbcagcagtc

SEQ ID NO: 65 - DNA, 20 bp, Artificial Sequence
  HB3 primer
  misc_feature (16)..(16): s is c or g
  misc_feature (18)..(18): s is c or g
caggtgcagctgaagsastc

SEQ ID NO: 66 - DNA, 20 bp, Artificial Sequence
  HB4 primer
  misc_feature (9)..(9): r is a or g
  misc_feature (18)..(18): r is a or g
gaggtccarctgcaacartc

SEQ ID NO: 67 - DNA, 20 bp, Artificial Sequence
  HB5 primer
  misc_feature (6)..(6): y is c or t
  misc_feature (12)..(12): b is g or c or t
  misc_feature (18)..(18): r is a or g
caggtycagctbcagcartc

SEQ ID NO: 68 - DNA, 20 bp, Artificial Sequence
  HB6 primer
  misc_feature (6)..(6): y is c or t
  misc_feature (9)..(9): r is a or g
caggtycarctgcagcagtc

SEQ ID NO: 69 - DNA, 20 bp, Artificial Sequence
  HB7 primer
caggtccacgtgaagcagtc

SEQ ID NO: 70 - DNA, 20 bp, Artificial Sequence
  HB8 primer
  misc_feature (9)..(10): s is c or g
gaggtgaasstggtggaatc

SEQ ID NO: 71 - DNA, 20 bp, Artificial Sequence
  HB9 primer
  misc_feature (3)..(3): v is a, c or g
  misc_feature (8)..(8): w is a or t
  misc_feature (10)..(10): y is c or t
gavgtgawgytggtggagtc

SEQ ID NO: 72 - DNA, 20 bp, Artificial Sequence
  HB10 primer
  misc_feature (10)..(10): s is c or g
  misc_feature (11)..(11): k is g or t
gaggtgcagskggtggagtc

SEQ ID NO: 73 - DNA, 20 bp, Artificial Sequence
  HB11 primer
  misc_feature (3)..(3): k is g or t
  misc_feature (9)..(9): m is a or c
gakgtgcamctggtggagtc

SEQ ID NO: 74 - DNA, 20 bp, Artificial Sequence
  HB12 primer
  misc_feature (18)..(18): r is a or g
gaggtgaagctgatggartc

SEQ ID NO: 75 - DNA, 20 bp, Artificial Sequence
  HB13 primer
  misc_feature (9)..(9): r is a or g
gaggtgcarcttgttgagtc

SEQ ID NO: 76 - DNA, 20 bp, Artificial Sequence
  HB14 primer
  misc_feature (3)..(3): r is a or g
  misc_feature (6)..(6): r is a or g
gargtraagcttctcgagtc

SEQ ID NO: 77 - DNA, 20 bp, Artificial Sequence
  HB15 primer
  misc_feature (9)..(9): r is a or g
  misc_feature (10)..(10): s is c or g
gaagtgaarsttgaggagtc

SEQ ID NO: 78 - DNA, 22 bp, Artificial Sequence
  HB16 primer
  misc_feature (12)..(12): r is a or g
  misc_feature (17)..(17): w is a or t
  misc_feature (20)..(20): s is c or g
caggttactctraaagwgtstg

SEQ ID NO: 79 - DNA, 20 bp, Artificial Sequence
  HB17 primer
  misc_feature (12)..(12): v is a or c
  misc_feature (18)..(18): r is a or g
caggtccaactvcagcarcc

SEQ ID NO: 80 - DNA, 20 bp, Artificial Sequence
  HB18 primer
gatgtgaacttggaagtgtc

SEQ ID NO: 81 - DNA, 20 bp, Artificial Sequence
  HB19 primer
gaggtgaaggtcatcgagtc

SEQ ID NO: 82 - DNA, 20 bp, Artificial Sequence
  HF1 primer
gaggaaacggtgaccgtggt

SEQ ID NO: 83 - DNA, 20 bp, Artificial Sequence
  HF2 primer
gaggagactgtgagagtggt

SEQ ID NO: 84 - DNA, 20 bp, Artificial Sequence
  HF3 primer
gcagagacagtgaccagagt

SEQ ID NO: 85 - DNA, 20 bp, Artificial Sequence
  HF4 primer
gaggagacggtgactgaggt

(19) United States
(12) Patent Application Publication
(10) Pub. No.: US 2007/0087331 A1
(43) Pub. Date: Apr. 19, 2007

(54) Selection of b cells with specificity if interest: method of preparation and use

(75) Inventors: Martin F. Bachmann, Seuzach (CH); Dominique Gatto, Sydney (AU)

Correspondence Address: STERNE, KESSLER, GOLDSTEIN & FOX P.L.L.C., 1100 NEW YORK AVENUE, N.W., WASHINGTON, DC, 20005, US

(73) Assignee: Cytos Biotechnology AG, Zurich-Schlieren (CH)

(21) Appl. No.: 10/556,904

(22) PCT Filed: May 14, 2004
(86) PCT No.: PCT/EP04/05208

§ 371 Date: Nov. 21, 2006

Related U.S. Application Data
(60) Provisional application No. 60/470,443, filed on May 15, 2003.

(51) Int. Cl. C12Q 1/70; C12N 5/20; G01N 33/567

(52) U.S. Cl. 435/5; 435/343.1; 435/7.2; 435/7.92; 977/802

(57) ABSTRACT

The present invention is related to the fields of molecular biology, virology, immunology and medicine. The invention provides methods using a composition comprising an ordered and repetitive antigen or antigenic determinant array for visualization and selection of B cells specific for the antigen. These B cells are useful for the production of monoclonal antibodies used for therapy, diagnostic or research purposes.

### BACKGROUND OF THE INVENTION

1. Field of the Invention

The present invention is related to the fields of vaccinology, monoclonal antibodies and medicine. The invention provides methods for selecting B cells with a desired specificity using virus-like particles decorated with an antigen of choice as detection device. RNA can be isolated from such B cells for the generation of cDNA encoding the variable regions of immunoglobulins encoded by B cells for recombinant production of monoclonal antibodies. Alternatively, B cells may be fused in vitro with a fusion partner, allowing the generation of hybridomas secreting monoclonal antibodies of the desired specificity. Such monoclonal antibodies may be used for research purposes, diagnostic purposes or the treatment of diseases.

2. Related Art

Monoclonal antibodies have become amongst the most efficient therapeutic regimens. Due to their enormous specificity and high affinity for the target structure recognized, monoclonal antibodies are ideal tools for diagnostic use or for use in research in general. In addition, due to their high specificity, monoclonal antibodies often exhibit a limited side-effect spectrum in vivo which makes them highly suitable for use in therapy. In addition, the mechanisms of action of monoclonal antibodies are manifold. On one hand, via their constant Fc parts, monoclonal antibodies interact with Fc receptors which allows for the recruitment of effector cells, such as natural killer (NK) cells or macrophages, to tissue recognized by the monoclonal antibodies, leading to destruction of the target tissue. This makes monoclonal antibodies powerful weapons against cancer. In addition, by radiolabeling monoclonal antibodies or by coupling toxins to monoclonal antibodies, the ability to destroy target tissues may be enhanced. With this regard, toxins that are activated only after cellular uptake seem to be particularly interesting. In addition, unlike most traditionally used small molecules, monoclonal antibodies are able to block protein-protein interactions in vivo, allowing to selectively interfere with the action of cytokines, chemokines, hormones etc. This unique feature of monoclonal antibodies is responsible for their broad usefulness in treating a vast number of diseases.

Generation of monoclonal antibodies has first been described by Kohler and Milstein (Kohler, G. and Milstein, C. (1975) Nature 256 (5517):495-7) using the hybridoma technology and since then has become a standard procedure in the lab. The hybridoma technology enables one to immortalite individual B cells, and thereby allows one to expand a population of individual B cell clones to obtain sufficient antibodies so that the immortalized population can be screened to isolate those B cells producing antibodies having a particular antigen specificity. The B cell hybrids of interest can be grown in a large scale to make large quantities of homogeneous monoclonal antibodies, which are useful for diagnostic and therapeutic purposes. Hybridoma technology generally works best for preparing purely murine monoclonal antibodies. Hybrids made from fusing human B cells with human or murine myeloma cells are generally unstable, and tend to lose the human chromosomes and the ability to produce antibodies. Transforming human B cells with the Epstein-Barr virus (EBV) offers an alternative way to immortalize them. However, the transformation frequency is relatively low and the transformants are also not stable and often lose antibody-producing ability.

Therefore, production of monoclonal antibodies has remained a tedious process that is very time consuming. Specifically, mice have to be multiply immunized before spleen cells are randomly fused with non-Ig secreting myeloma cells leading to immortalization of the B cell fusion partner. However, since specific B cells are not extensively enriched in this process, many hybridomas have to be screened, subcloned, and re-screened before specific monoclonal antibodies may be isolated. In addition, only monoclonal antibodies exhibiting a high affinity are interesting for most purposes. However, before the affinity of the antibodies can be assessed, the cells producing these antibodies need to be clonal. This makes it necessary that many more hybridomas have to be generated, analysed subcloned etc than would really be necessary, since the affinity of the monoclonal antibodies can only be determined definitively at the final step.

A further problem is that the diversity of the immune system means that lymphocytes recognizing a particular antigen are rare. Estimations of the frequencies of cells specific for one antigen used to range from 10-5 to 10-6, based for example on limiting dilution analyses. For a number of biological and physical reasons immunofluorescence, either with antigens or antibodies, shows considerable variation in intensity. This makes it technically difficult to identify accurately rare cells of interest at frequencies below 10-3 to 10-4. Apart from that basic limitation, it is extremely time consuming to analyze a sufficient number of rare cells to obtain a reliable result. In addition, the cytometry of B lymphocytes according to antigen specificity is a problem of biology, because B cells that bind to one particular antigen often occur at frequencies of 1-10/ml blood, thus making the availability of sufficient blood for analysis a limiting factor.

Various methods have been employed to enrich the desired specific B cells when they occur at low frequency. These methods can be used to enrich the desired B cells before directly isolating RNA and cDNA encoding the variable regions of the monoclonal antibodies of interest whether one is attempting to isolate these fragments by hybridoma fusion technology, EBV transformation, or the combinatorial library methodology. These enrichment methods include fluorescence-activated cell sorting (FACS) (Dangl and Herzenberg (1982), J. Immunol. Methods 52, 1-14; Hoven et al. (1989), J. Immunol. Methods 117, 275-284), panning against antigen-coated plastic surface, binding to antigen-coated magnetic beads, or resetting with antigen-coated red blood cells.

These procedures for enriching antigen-specific B cells, however, all suffer from a certain degree of nonspecificity and background noise. For example, panning or absorbing to plates or beads yields from about one to several percent of nonspecific binding. Rosetting of cells by antigen-coated red blood cells also has about the same degree of nonspecific activity. For cell sorting using FACS, the nonspecific sorting will depend on the stringency of the gate setting, but it usually ranges from 0.1 to several percent depending on the nature of the antigen. Background noise in an FACS apparatus arises primarily from two sources. One source of noise is exhibited by certain activated cells or proliferating cells of various leukocyte subpopulations that contain high concentrations of certain metabolites, which cause these cells to exhibit autofluorescence even in the absence of fluorochrome labeling. Another possible source of background noise arises because some of the activated cells, and some monocytes and macrophages, possess sticky cellular plasmamembranes. Fluorescent probes will non-specifically adhere to the sticky plasmamembrane and create an additional source of background fluorescence in the cell sample. Furthermore, background noise can be caused by the presence of antibodies, specific for the immunizing/boosting antigen, which are present in the spleen cell suspension of immunized mice and which form immune complexes with the antigen-fluorochrome and bind to Fc receptors present on all B cells.

To overcome the problem of nonspecificity and background noise various methods have been developed that use for example at least two antigen probes that are labeled with different fluorochromes to label and specifically select B cells expressing antibodies of high affinity for the antigen of interest using the fluorescence-sorting technique FACS (U.S. Pat. No. 5,256,542). Another example is the use of ‘parallel’ cell-sorting technology (MACS), providing a nonoptical (in this case magnetic) label to enrich antigen-binding cells to make them detectable by flow cytometry and to isolate them for proof of specificity (Irsch et al., (1995), Immunotechnology, 1(2):115-25).

However, all the above described methods lack a certain degree of efficiency and specificity and further lack the differentiation between the selection for high affinity antigen-specific B cells and low affinity antigen-specific B cells. In particular, the methods known in the art all have the disadvantage that many hybridoma cells have to be generated, analyzed, subcloned, and re-screened before specific monoclonal antibodies may be isolated. This makes the known methods very time consuming and the affinity of the monoclonal antibodies can only be determined definitively at the final step. Thus, efficient methods for the selection of specific B cells are needed in the field in order to facilitate and hasten the production of monoclonal antibodies. Particularly, a method is needed that allows identification of monoclonal antibodies prior to the generation of hybridoma cells. In addition, a method is needed that allows specific detection, selection and isolation of B cells that express high affinity monoclonal antibodies.

### SUMMARY OF THE INVENTION

The present invention provides a method for the visualization, characterization and isolation of single antigen-specific B cells which are in particularly useful to facilitate and hasten the production of monoclonal antibodies, in particular of high affinity monoclonal antibodies, for diagnostic or therapeutic use.

The present invention provides methods based on compositions exhibiting antigens specifically coupled to them that allow detection and isolation of B cells specifically binding an antigen of choice. The compositions used in the present invention comprise antigens or antigenic determinants of interest which are bound to a core particle having a structure with an inherent repetitive organization, and hereby in particular to virus-like-particles (VLP's) and subunits of VLP's, respectively, leading to highly ordered and repetitive conjugates representing potent immunogens for the induction of antibodies specific for the antigen of interest. In particular, the compositions used in the method of the present invention allow for the detection, selection and isolation of either all antigen-specific B cells or of high affinity antigen-specific B cells. These selected B cells may be used for the production of monoclonal antibodies by means of generating hybridomas or isolating the genetic elements encoding the immunoglobulins expressed by the B cell. Thus, the present invention provides efficient methods for the isolation of single specific B cells which are used to facilitate and hasten the production of monoclonal antibodies. Particularly, the method allows the identification of monoclonal antibodies prior to the generation of hybridoma cells.

The present invention, thus, provides a method for selecting at least one antigen-specific B cell from a mixture of cells, said method comprising: (A) providing a mixture of cells comprising B cells; (B) providing a first composition comprising: (a) a first core particle with at least one first attachment site; and (b) at least one antigen or antigenic determinant with at least one second attachment site, wherein said second attachment site being selected from the group consisting of: (i) an attachment site not naturally occurring with said antigen or antigenic determinant; and (ii) an attachment site naturally occurring with said antigen or antigenic determinant, wherein said second attachment site is capable of association to said first attachment site; and wherein said antigen or antigenic determinant and said first core particle interact through said association to form an ordered and repetitive antigen array; (C) contacting said mixture of cells with said first composition; (D) labeling said first composition with a first labeling compound; (E) labeling said B cells in said mixture of cells with a second labeling compound; and (F) selecting at least one B cell which is positive for said first and said second labeling compound. Preferred embodiments of first core particles suitable for use in the present invention are a virus, a virus-like particle, in particular a Hepatitis B virus core antigen, a bacteriophage, a bacterial pilus or flagella, a viral capsid particle, a virus-like particle (VLP) of a RNA-phage, in particular a VLP of the RNA phage Qβ, or any recombinant form of the aforementioned first core particles, or any other core particle having an inherent repetitive structure capable of forming an ordered and repetitive antigen array in accordance with the present invention.

WO 00/32227, WO01/85208, and in particular WO 02/056905 describe compositions and processes for the production of ordered and repetitive antigen or antigenic determinant arrays. The compositions comprise a core particle, such as a virus or a virus-like particle, to which at least one antigen or one antigenic determinant, is associated by way of at least one non-peptide bond leading to the ordered and repetitive antigen array. Virus-like particles (VLPs) are supermolecular structures built in a symmetric manner from many protein molecules of one or more types. They lack the viral genome and, therefore, are noninfectious. VLPs can often be produced in large quantities by heterologous expression and can be easily purified. Examples of VLPs may include, without limitation, the capsid proteins of Hepatitis B virus (Ulrich, et al., Virus Res. 50:141-182 (1998)), measles virus (Warnes, et al., Gene 160:173-178 (1995)), Sindbis virus, rotavirus (U.S. Pat. No. 5,071,651 and U.S. Pat. No. 5,374,426), foot-and-mouth-disease virus (Twomey, et al., Vaccine 13:1603 1610, (1995)), Norwalk virus (Jiang, X., et al., Science 250:1580 1583 (1990); Matsui, S. M., et al., J. Clin. Invest. 87:1456 1461 (1991)), the retroviral GAG protein (WO 96/30523), the retrotransposon Ty protein p1, the surface protein of Hepatitis B virus (WO 92/11291) and human papilloma virus (WO 98/15631).

VLPs exhibit the same structure as viruses. Most viruses are particulate structures and exhibit a highly repetitive surface. Due to the repetitiveness of their surface, viruses are able to efficiently cross-link B cell receptors and trigger strong B cell responses. In addition, as previously shown, viruses may bind with high efficiency to specific B cells allowing to selectively detect anti-viral B cells in histology (Bachmann, M F. et al., (1996) J Exp Med., 183(5):2259-69) or using flowcytometry (Youngman, K R. Et al., (2002) J Immunol. 168(5):2173-81). The detection of bound virus was feasible since the viruses served as a signal amplification tool; one virus binding to a B cell exhibits several hundred epitopes that may be detected by secondary reagents.

In one aspect of the present invention, the second attachment site of the first composition used for the method of the invention is capable of association to said first attachment site through at least one covalent bond, preferably through at least one non-peptide bond.

The first composition used for the method of the invention may comprise an antigen or antigenic determinant that may be a recombinant antigen or a synthetic peptide. In particular, it may be selected from the group consisting of polypeptides, carbohydrates, steroid hormones, organic molecules, inorganic molecules, viruses, bacteria, parasites, prions, tumors, self-molecules, non-peptide hapten molecules, and allergens. In one aspect of the present invention, the antigen or antigenic determinant is attached to said first core particle at high density allowing efficient selection of all antigen-specific B cells. In another aspect of the present invention, the antigen or antigenic determinant is attached to said first core particle at low density allowing for specific selection of high affinity antigen-specific B cells.

In one embodiment of the present invention, said selecting at least one B cell which is positive for said first and said second labeling compound is effected by using a first device capable of detecting said first labeling compound and a second device capable of detecting said second labeling compound.

In a preferred embodiment, said first labeling compound is a first fluorochrome and said second labeling compound is a second fluorochrome, said first fluorochrome yielding a color different from said second fluorochrome upon activation, and wherein said first device capable of detecting said first labeling compound and said second device capable of detecting said second labeling compound is a fluorescence activated cell sorting apparatus (FACS).

In a further aspect of the present invention, said labeling of said first composition is effected prior to contacting said mixture with said first composition. In another aspect of the invention, said labeling of said first composition is effected after contacting said mixture with said first composition, preferably with at least one antibody probe, wherein said probe comprises an antibody which is specific for said first composition, preferably specific for said core particle of said first composition, said antibody being conjugated with said first labeling compound. In another preferred embodiment, said labeling effected after contacting said mixture with said first composition is effected with at least one first antibody probe, wherein said first probe comprises a first antibody which is specific for said first composition, preferably specific for said first core particle of said first composition, and at least one second antibody probe, wherein said second probe comprises a second antibody which is specific for said first antibody, said second antibody being conjugated with said first labeling compound.

In a further aspect, said labeling said B cells in said mixture of cells with a second labeling compound may be effected with a first set of at least one additional first targeting molecule, wherein said first targeting molecule is specific for at least one B cell marker, and wherein said first targeting molecule is labeled with a second labeling compound.

In still a further embodiment, the method of the present invention further comprises labeling said mixture with a second set of at least one second additional targeting molecule, wherein said at least one second targeting molecule is specific for at least one marker unique for cells other than isotype-switched B cells, and wherein said at least one second targeting molecule is labeled with a third labeling compound.

In yet another aspect of the invention, the method of the invention further comprises an additional step of adding a dead cell marker to said mixture of cells.

The method of the present invention further comprises the step of verifying specific antibody production of said selected at least one antigen-specific B cell. In a preferred embodiment, said verifying is effected by ELISA.

In a preferred aspect of the method of the present invention, said mixture of cells is a mixture of splenocytes from immunized animals, said animals being immunized with a second composition comprising: (a) a second core particle with at least one first attachment site; and (b) at least one antigen or antigenic determinant with at least one second attachment site, wherein said second attachment site being selected from the group consisting of: (i) an attachment site not naturally occurring with said antigen or antigenic determinant; and (ii) an attachment site naturally occurring with said antigen or antigenic determinant, wherein said second attachment site is capable of association to said first attachment site; wherein said antigen or antigenic determinant and said second core particle interact through said association to form an ordered and repetitive antigen array. In a preferred embodiment, said second core particle is different from said first core particle. In a further embodiment, said second core particle is the same as said first core particle. Preferred embodiments of second core particles suitable for use in the present invention are a virus, a virus-like particle, in particular a Hepatitis B virus core antigen, a bacteriophage, a bacterial pilus or flagella, a viral capsid particle, a virus-like particle (VLP) of a RNA-phage, in particular a VLP of the RNA-phage Qβ, or any recombinant form of the aforementioned first core particles, or any other core particle having an inherent repetitive structure capable of forming an ordered and repetitive antigen array in accordance with the present invention. In a preferred embodiment, the second composition used for the method of the invention comprises the same antigen or antigenic determinant as the antigen or antigenic determinant of the first composition. In another embodiment, the second composition used for the method of the invention comprises a different antigen or antigenic determinant as the antigen or antigenic determinant of the first composition.

The present invention further provides an antigen-specific B cell selected by any of the methods of the invention.

In a further aspect, the present invention provides a method for generating monoclonal antibodies, comprising providing at least one antigen-specific B cell selected by any of the methods of the invention and fusing said at least one antigen-specific B cell with a myeloma cell line. In a preferred embodiment, the antigen-specific B cell used for fusing with a myeloma cell line is selected from a mixture of cells from immunized animals.

In another embodiment, the present invention provides a method for generating monoclonal antibodies or antibody fragments comprising isolating genetic elements encoding the immunoglobulin or parts of the immunoglobulin expressed by said at least one antigen-specific B cell selected by a method of the invention and further expressing said genetic elements.

It is to be understood that both the foregoing general description and the following detailed description are exemplary and explanatory only and are intended to provide further explanation of the invention as claimed.

### BRIEF DESCRIPTION OF THE DRAWINGS/FIGURES

FIG. 1 shows FACS staining of specific B cells with VLPs and anti-VLP antiserum. Lymphocytes were selected on the basis of their forward and side scatter and dead cells (PI+) were excluded. Isotype-switched B cells (CD19+IgM−IgD−) were distingushed from naive B cells, T cells, macrophages and granulocytes with a negative selection step, in which IgM+, IgD+, CD4+, CD8+ CD11b+ and Gr-1+ cells were gated out. Switched B cells were analysed for Qβ-binding.

FIG. 2 shows staining of specific B cells with labelled VLPs. Lymphocytes were selected on the basis of their forward and side scatter. Isotype-switched B cells (CD19+IgM−IgD−) were distingushed from naive B cells, T cells, macrophages, granulocytes and dead cells with a negative selection step, in which IgM+, IgD+, CD4+, CD8+, CD11b+, Gr-1+ and PI+ cells were excluded. Switched B cells were analysed for Qβ-binding.

FIG. 3 shows coupling of peptide to Qβ with high (A) or low (B) efficiency and to HBcAg (C).

FIG. 4 shows FACS staining of specific B cells with peptide-coupled VLPs. Isotype-switched B cells were identified as in FIG. 1 and binding to D2-peptide coupled with high (left) or low (middle) efficiency to Qβ, as well as binding to uncoupled Qβ(right), was assessed. High affinity peptide-specific B cells were selected using Qβ exhibiting few peptides coupled to it. Background staining in unimmunised mice is shown (lower panels).

FIG. 5 shows an analysis of the families of V (FIG. 5A), D (FIG. 5B), and J (FIG. 5C) segments of heavy chain variable region sequences amplified from sorted Qβ-specific B cells. V region genes have been grouped into families, whose members share about 80% identity at the DNA level. Sequences of V_{H} segments cloned from purified Qβ-specific B cells were determined and the families of the V, D, and J segments were assessed by matching the sequences against a database of murine immunoglobulin germline sequences. The internet site used for the quest was: http://imgt.cines.fr. This analysis allows for the determination of the diversity of the cloned antibody sequences. The number of isolated sequences belonging to a specific family (immunoglobulin heavy variable (IGHV) subgroup 1-14 for the V segment (FIG. 5A), immunoglobulin heavy diversity (IGHD) subgroup 1-4 for the D segment (FIG. 5B), and immunoglobulin heavy joining (IGHJ) subgroup 1-4 for the J segment (FIG. 5C)) is depicted in the figure. As shown, the cloned antibody fragments could be assigned to various V, D or J families.

### DETAILED DESCRIPTION OF THE INVENTION

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods and materials are hereinafter described.

1. Definitions

Amino acid linker: An “amino acid linker”, or also just termed “linker” within this specification, as used herein, either associates the antigen or antigenic determinant with the second attachment site, or more preferably, already comprises or contains the second attachment site, typically—but not necessarily—as one amino acid residue, preferably as a cysteine residue. The term “amino acid linker” as used herein, however, does not intend to imply that such an amino acid linker consists exclusively of amino acid residues, even if an amino acid linker consisting of amino acid residues is a preferred embodiment of the present invention. The amino acid residues of the amino acid linker are, preferably, composed of naturally occuring amino acids or unnatural amino acids known in the art, all-L or all-D or mixtures thereof. However, an amino acid linker comprising a molecule with a sulfhydryl group or cysteine residue is also encompassed within the invention. Such a molecule comprise preferably a C1-C6 alkyl-, cycloalkyl (C5,C6), aryl or heteroaryl moiety. However, in addition to an amino acid linker, a linker comprising preferably a C1-C6 alkyl-, cycloalkyl-(C5,C6), aryl- or heteroaryl-moiety and devoid of any amino acid(s) shall also be encompassed within the scope of the invention. Association between the antigen or antigenic determinant or optionally the second attachment site and the amino acid linker is preferably by way of at least one covalent bond, more preferably by way of at least one peptide bond.

Animal: As used herein, the term “animal” is meant to include, for example, humans, sheep, horses, cattle, pigs, dogs, cats, rats, mice, birds, reptiles, fish, insects and arachnids.

Antibody: As used herein, the term “antibody” refers to molecules which are capable of binding an epitope or antigenic determinant. The term is meant to include whole antibodies and antigen-binding fragments thereof including single-chain antibodies. Most preferably the antibodies are human antigen binding antibody fragments and include, but are not limited to, Fab, Fab′ and F(ab′)2, Fd, single-chain Fvs (scFv), single-chain antibodies, disulfide-linked Fvs (sdFv) and fragments comprising either a VL or VH domain. The antibodies can be from any animal origin including birds and mammals. Preferably, the antibodies are human, murine, rabbit, goat, guinea pig, camel, horse or chicken. As used herein, “human” antibodies include antibodies having the amino acid sequence of a human immunoglobulin and include antibodies isolated from human immunoglobulin libraries or from animals transgenic for one or more human immunoglobulins and that do not express endogenous immunoglobulins, as described, for example, in U.S. Pat. No. 5,939,598 by Kucherlapati et al.

Antigen: As used herein, the term “antigen” refers to a molecule capable of being bound by an antibody or a T cell receptor (TCR) if presented by MHC molecules. The term “antigen”, as used herein, also encompasses T-cell epitopes. An antigen is additionally capable of being recognized by the immune system and/or being capable of inducing a humoral immune response and/or cellular immune response leading to the activation of B- and/or T-lymphocytes. This may, however, require that, at least in certain cases, the antigen contains or is linked to a Th cell epitope and is given in adjuvant. An antigen can have one or more epitopes (B- and T-epitopes). The specific reaction referred to above is meant to indicate that the antigen will preferably react, typically in a highly selective manner, with its corresponding antibody or TCR and not with the multitude of other antibodies or TCRs which may be evoked by other antigens. Antigens as used herein may also be mixtures of several individual antigens.

A “microbial antigen” as used herein is an antigen of a microorganism and includes, but is not limited to, infectious virus, infectious bacteria, parasites and infectious fungi. Such antigens include the intact microorganism as well as natural isolates and fragments or derivatives thereof and also synthetic or recombinant compounds which are identical to or similar to natural microorganism antigens and induce an immune response specific for that microorganism. A compound is similar to a natural microorganism antigen if it induces an immune response (humoral and/or cellular) to a natural microorganism antigen. Such antigens are used routinely in the art and are well known to the skilled artisan.

Antigenic determinant: As used herein, the term “antigenic determinant” is meant to refer to that portion of an antigen that is specifically recognized by either B- or T-lymphocytes. B-lymphocytes respond to foreign antigenic determinants via antibody production, whereas T-lymphocytes are the mediator of cellular immunity. Thus, antigenic determinants or epitopes are those parts of an antigen that are recognized by antibodies, or in the context of an MHC, by T-cell receptors.

Antigen presenting cell: As used herein, the term “antigen presenting cell” is meant to refer to a heterogenous population of leucocytes or bone marrow derived cells which possess an immunostimulatory capacity. For example, these cells are capable of generating peptides bound to MHC molecules that can be recognized by T cells. The term is synonymous with the term “accessory cell” and includes, for example, Langerhans' cells, interdigitating cells, B cells and macrophages. Under some conditions, epithelial, endothelial cells and other, non-bone marrow derived cells may also serve as antigen presenting cells.

Association: As used herein, the term “association” as it applies to the first and second attachment sites, refers to the binding of the first and second attachment sites that is preferably by way of at least one non-peptide bond. The nature of the association may be covalent, ionic, hydrophobic, polar or any combination thereof, preferably the nature of the association is covalent, and again more preferably the association is through at least one, preferably one, non-peptide bond. As used herein, the term “association” as it applies to the first and second attachment sites, not only encompass the direct binding or association of the first and second attachment site forming the compositions of the invention but also, alternatively and preferably, the indirect association or binding of the first and second attachment site leading to the compositions of the invention, and hereby typically and preferably by using a heterobifunctional cross-linker.

Attachment Site, First: As used herein, the phrase “first attachment site” refers to an element of non-natural or natural origin, typically and preferably being comprised by the virus-like particle, to which the second attachment site typically and preferably being comprised by the antigen or antigenic determinant may associate. The first attachment site may be a protein, a polypeptide, an amino acid, a peptide, a sugar, a polynucleotide, a natural or synthetic polymer, a secondary metabolite or compound (biotin, fluorescein, retinol, digoxigenin, metal ions, phenylmethylsulfonylfluoride), or a combination thereof, or a chemically reactive group thereof. The first attachment site is located, typically and preferably on the surface, of the core particle such as, preferably the virus-like particle. Multiple first attachment sites are present on the surface of the core and virus-like particle, respectively, typically in a repetitive configuration. Preferably, the first attachment site is a amino acid or a chemically reactive group thereof.

Attachment Site, Second: As used herein, the phrase “second attachment site” refers to an element associated with, typically and preferably being comprised by, the antigen or antigenic determinant to which the first attachment site located on the surface of the core particle and virus-like particle, respectively, may associate. The second attachment site of the antigen or antigenic determinant may be a protein, a polypeptide, a peptide, a sugar, a polynucleotide, a natural or synthetic polymer, a secondary metabolite or compound (biotin, fluorescein, retinol, digoxigenin, metal ions, phenylmethylsulfonylfluoride), or a combination thereof, or a chemically reactive group thereof. At least one second attachment site is present on the antigen or antigenic determinant. The term “antigen or antigenic determinant with at least one second attachment site” refers, therefore, to an antigen or antigenic construct comprising at least the antigen or antigenic determinant and the second attachment site. However, in particular for a second attachment site, which is of non-natural origin, i.e. not naturally occurring within the antigen or antigenic determinant, these antigen or antigenic constructs comprise an “amino acid linker”.

B cell: As used herein, the term “B cell” refers to a cell produced in the bone marrow of an animal expressing membrane-bound antibody specific for an antigen. Following interaction with the antigen it differentiates into a plasma cell producing antibodies specific for the antigen or into a memory B cell. “B cell” and “B lymphocyte” is used interchangeably. Naïve as well as activated B cells are within the scope of the invention.

Antigen-specific B cell: As used herein, the term “antigen-specific B cell” refers to a B cell which expresses antibodies that are able to distinguish between the antigen of interest and other antigens and which specifically bind to that antigen of interest with high or low affinity but which do not bind to other antigens.

Positive B cell: As used herein, the term “positive B cell” means any B cell which is labeled with any one of the labeling compounds of the invention and which is selected or sorted or otherwise separated from a mixture of cells by a device capable of detecting said labeling compound. For example, a B cell which is positive for the first labeling compound of the invention is a B cell which is labeled with a first labeling compound and which is selected by the device capable of detecting said first labeling compound.

Switched B cell: As used herein, the term “switched B cell” refers to activated B cells which have undergone isotype switching or class switching to secrete antibodies of different isotypes: IgG, IgA, and IgE. Isotype switching does not affect antibody specificity significantly, but alters the effector functions that an antibody can engage. Isotype switching occurs by recombination involving the deletion of DNA between the rearranged V region and the selected C-region exon at so-called S regions (see Janeway et al., Immunobiology, 5th edition, 2001, New York and London, Garland Publishing). Markers for cells other than switched B cells suitable for use in the present invention include, but are not limited to IgD, IgM, CD2, CD3, CD4, CD8, CD11b, Gr-1, Thy-1, CD43.

B cell marker: As used herein, the term “B cell marker” refers to surface molecules on the B cells which are specific for antigen-specific IgG-producing B cells. B cell markers suitable for use in the present invention include, but are not limited to surface IgG, kappa and lambda chains, Ig-alpha (CD79alpha), Ig-beta (CD79beta), CD19, Ia, Fc receptors, B220 (CD45R), CD20, CD21, CD22, CD23, CD81 (TAPA-1) or any other CD antigen specific for B cells.

Bound: As used herein, the term “bound” refers to binding or attachment that may be covalent, e.g., by chemically coupling, or non-covalent, e.g., ionic interactions, hydrophobic interactions, hydrogen bonds, etc. Covalent bonds can be, for example, ester, ether, phosphoester, amide, peptide, imide, carbon-sulfur bonds, carbon-phosphorus bonds, and the like. The term “bound” is broader than and includes terms such as “coupled”, “fused”, “associated” and “attached”.

Coat protein(s): As used herein, the term “coat protein(s)” refers to the protein(s) of a bacteriophage or a RNA-phage capable of being incorporated within the capsid assembly of the bacteriophage or the RNA-phage. However, when referring to the specific gene product of the coat protein gene of RNA-phages the term “CP” is used. For example, the specific gene product of the coat protein gene of RNA-phage Qβ is referred to as “QβCP”, whereas the “coat proteins” of bacteriophage Qβ comprise the “Qβ CP” as well as the A1 protein. The capsid of Bacteriophage Qβ is composed mainly of the Qβ CP, with a minor content of the A1 protein. Likewise, the VLP Qβ coat protein contains mainly QβCP, with a minor content of A1 protein.

Core particle: As used herein, the term “core particle” refers to a rigid structure with an inherent repetitive organization. A core particle as used herein may be the product of a synthetic process or the product of a biological process.

Coupled: The term “coupled”, as used herein, refers to attachment by covalent bonds or by strong non-covalent interactions, typically and preferably to attachment by covalent bonds. Moreover, with respect to the coupling of the antigen to the virus-like particle the term “coupled” preferably refers to association and attachment, respectively, by at least one non-peptide bond. Any method normally used by those skilled in the art for the coupling of biologically active materials can be used in the present invention.

Dead cell marker: As used herein, the term “dead cell marker” refers to markers which specifically label dead cells. Dead cell markers suitable for use in the present invention include, but are not limited to propidium iodide (PI), YO Pro1 (YO-PRO®-1 iodide (491/509)), 7-AAD (7-aminoactinomycin D), EMA (ethidium monoazide bromide), BIS-Oxonol, To-Pro-3 (see Molecular Probes (Cat No T-3605)), RB2Z.

High density: As used herein, the term “high density” refers to high amounts of antigen presented on the surface of a core particle, preferably more than 0.7 antigens per subunit, more preferably more than 1 antigen per subunit.

Low density: As used herein, the term “low density” refers to low amounts of antigen presented on the surface of a core particle, preferably less than 0.5 antigens per subunit, more preferably less than 0.3 antigens per subunit.

Device capable of detecting: As used herein, the term “device capable of detecting” refers to a device or an apparatus that is capable of detecting, or otherwise identifying a labeling compound, such as e.g. a fluorochrome, or a magnetic particle. A device capable of detecting a fluorochrome may include without limitation a fluorescence activated cell sorting apparatus (FACS). A device capable of detecting a magnetic particle may include without limitation a magnetic cell sorting apparatus (MACS).

Epitope: As used herein, the term “epitope” refers to continuous or discontinuous portions of a polypeptide having antigenic or immunogenic activity in an animal, preferably a mammal, and most preferably in a human. An epitope is recognized by an antibody or a T cell through its T cell receptor in the context of an MHC molecule. An “immunogenic epitope,” as used herein, is defined as a portion of a polypeptide that elicits an antibody response or induces a T-cell response in an animal, as determined by any method known in the art. (See, for example, Geysen et al., Proc. Natl. Acad. Sci. USA 81:3998 4002 (1983)). The term “antigenic epitope,” as used herein, is defined as a portion of a protein to which an antibody can immunospecifically bind its antigen as determined by any method well known in the art. Immunospecific binding excludes non specific binding but does not necessarily exclude cross reactivity with other antigens. Antigenic epitopes need not necessarily be immunogenic. Antigenic epitopes can also be T-cell epitopes, in which case they can be bound immunospecifically by a T-cell receptor within the context of an MHC molecule.

An epitope can comprise 3 amino acids in a spatial conformation which is unique to the epitope. Generally, an epitope consists of at least about 5 such amino acids, and more usually, consists of at least about 8-10 such amino acids. If the epitope is an organic molecule, it may be as small as Nitrophenyl.

Fusion: As used herein, the term “fusion” refers to the combination of amino acid sequences of different origin in one polypeptide chain by in-frame combination of their coding nucleotide sequences. The term “fusion” explicitly encompasses internal fusions, i.e., insertion of sequences of different origin within a polypeptide chain, in addition to fusion to one of its termini.

Immune response: As used herein, the term “immune response” refers to a humoral immune response and/or cellular immune response leading to the activation or proliferation of B- and/or T-lymphocytes and/or and antigen presenting cells. In some instances, however, the immune responses may be of low intensity and become detectable only when using at least one substance in accordance with the invention. “Immunogenic” refers to an agent used to stimulate the immune system of a living organism, so that one or more functions of the immune system are increased and directed towards the immunogenic agent. An “immunogenic polypeptide” is a polypeptide that elicits a cellular and/or humoral immune response, whether alone or linked to a carrier in the presence or absence of an adjuvant. Preferably, antigen presenting cell may be activated.

A substance which “enhances” an immune response refers to a substance in which an immune response is observed that is greater or intensified or deviated in any way with the addition of the substance when compared to the same immune response measured without the addition of the substance. For example, the lytic activity of cytotoxic T cells can be measured, e.g. using a 51Cr release assay, typically and preferably as outlined in Current Protocols in Immunology, Editors: John E. Coligan et al.; John Wiley & Sons Inc., with and without the substance. The amount of the substance at which the CTL lytic activity is enhanced as compared to the CTL lytic activity without the substance is said to be an amount sufficient to enhance the immune response of the animal to the antigen. In a preferred embodiment, the immune response in enhanced by a factor of at least about 2, more preferably by a factor of about 3 or more. The amount or type of cytokines secreted may also be altered. Alternatively, the amount of antibodies induced or their subclasses may be altered.

Immunization: As used herein, the terms “immunize” or “immunization” or related terms refer to conferring the ability to mount a substantial immune response (comprising antibodies and/or cellular immunity such as effector CTL) against a target antigen or epitope. These terms do not require that complete immunity be created, but rather that an immune response be produced which is substantially greater than baseline. For example, a mammal may be considered to be immunized against a target antigen if the cellular and/or humoral immune response to the target antigen occurs following the application of methods of the invention.

Labeling compound: As used herein, the term “labeling compound” refers to a compound used to label the first composition, the B cell markers, or markers for cells other than switched B cells of the invention either directly or indirectly through, for example, a tag, antibody, dioxigenin, or biotin. Such labels suitable for use in the present invention are well known in the art and include, but are not limited to fluorescent materials (e.g. PerCP, Allophycocyanin (APC), texas red, rhodamine, Cy3, Cy5, Cy5.5, Cy7, Alexa Fluor Dyes, phycoerythrin (PE), green fluorescent protein (GFP), a tandem dye (e.g. PE-Cy5), fluorescein isothiocyanate (FITC)), magnetic beads, radiolabel (e.g. 131I-labeled antibody, 90Y (a pure beta emitter)-labeled antibody, 211At-labeled antibody), an enzyme, avidin or biotin, or any other tag or label known in the art useful for labeling the composition of the invention. The first composition of the invention may be labeled prior to or after contacting said mixture of cells with said first composition. If said first composition is labeled prior to contacting said mixture of cells with said first composition, said first composition is labeled with a first labeling compound selected from the group consisting of without limitation avidin or biotin, dioxigenin, flag tag or any other tag known in the art. In addition, to detect said first labeling compound, the B cell in the mixture of cells is labeled after contactin said mixture of cells with labeled streptavidin, anti-dioxigenin, anti-flag or any other anti-tag. The first composition of the invention may also be labeled after contacting said mixture of cells with said first composition with a first antibody specific for said first composition, specific for the B cell markers, or specific for markers for cells other than switched B cells of the invention, said first antibody being labeled with labels selected from the group consisting of without limitation fluorescent materials, magnetic particles or radiolabels. Alternatively, said first composition may further be labeled after contacting the mixtures of cells with said first composition with a first antibody specific for said first composition, specific for the B cell markers, or specific for markers for cells other than switched B cells of the invention, and further with a second antibody specific for said first antibody, said second antibody being labeled with labels selected from the group consisting of without limitation fluorescent materials, magnetic particles or radiolabels. In a preferred embodiment, the first, second, and third fluorochrome of the invention may yield different colors upon activation.

Mixture of cells: As used herein, the term “mixture of cells” refers to any mixture of cells comprising B cells, preferably to a cell suspension of cells of the peripheral lymphoid organs or of peripheral blood cells. Cells of the peripheral lymphoid organs may include without limitation splenocytes from the spleen or lymphocytes from lymph nodes. Cells used for the method of the present invention are from animals, preferably mammals, even more preferably from mammals immunized with an antigen of interest, preferably immunized with the second composition of the invention.

Natural origin: As used herein, the term “natural origin” means that the whole or parts thereof are not synthetic and exist or are produced in nature.

Non-natural: As used herein, the term generally means not from nature, more specifically, the term means from the hand of man.

Non-natural origin: As used herein, the term “non-natural origin” generally means synthetic or not from nature; more specifically, the term means from the hand of man.

Ordered and repetitive antigen or antigenic determinant array: As used herein, the term “ordered and repetitive antigen or antigenic determinant array” generally refers to a repeating pattern of antigen or antigenic determinant, characterized by a uniform spacial arrangement of the antigens or antigenic determinants with respect to the core particle. In one embodiment of the invention, the repeating pattern may be a geometric pattern. Examples of suitable ordered and repetitive antigen or antigenic determinant arrays are those which possess strictly repetitive paracrystalline orders of antigens or antigenic determinants, preferably with spacings of 0.5 to 30 nanometers, more preferably 3 to 15 nanometers, even more preferably 3 to 8 nanometers.

Pili: As used herein, the term “pili” (singular being “pilus”) refers to extracellular structures of bacterial cells composed of protein monomers (e.g., pilin monomers) which are organized into ordered and repetitive patterns. Further, pili are structures which are involved in processes such as the attachment of bacterial cells to host cell surface receptors, inter cellular genetic exchanges, and cell cell recognition. Examples of pili include Type 1 pili, P pili, F1C pili, S pili, and 987P pili. Additional examples of pili are set out below.

Pilus like structure: As used herein, the phrase “pilus-like structure” refers to structures having characteristics similar to that of pili and composed of protein monomers. One example of a “pilus-like structure” is a structure formed by a bacterial cell which expresses modified pilin proteins that do not form ordered and repetitive arrays but that are essentially identical to those of natural pili. Another example for a “pilus-like structure” is a structure formed by a bacterial cell which expresses modified pilin proteins that do form ordered and repetitive arrays but that have different symmetrical properties compared to those of natural pili.

Polypeptide: As used herein, the term “polypeptide” refers to a molecule composed of monomers (amino acids) linearly linked by amide bonds (also known as peptide bonds). It indicates a molecular chain of amino acids and does not refer to a specific length of the product. Thus, peptides, oligopeptides and proteins are included within the definition of polypeptide. This term is also intended to refer to post-expression modifications of the polypeptide, for example, glycosolations, acetylations, phosphorylations, and the like. A recombinant or derived polypeptide is not necessarily translated from a designated nucleic acid sequence. It may also be generated in any manner, including chemical synthesis.

Residue: As used herein, the term “residue” is meant to mean a specific amino acid in a polypeptide backbone or side chain.

Self antigen: As used herein, the term “self antigen” refers to proteins encoded by the host's DNA and products generated by proteins or RNA encoded by the host's DNA are defined as self. Preferably, the term “self antigen”, as used herein, refers to proteins encoded by the human genome or DNA and products generated by proteins or RNA encoded by the human genome or DNA are defined as self. The inventive compositions, pharmaceutical compositions and vaccines comprising self antigens are in particular capable of breaking tolerance against a self antigen when applied to the host. In this context, “breaking tolerance against a self antigen” shall refer to enhancing an immune response, as defined herein, and preferably enhancing a B or a T cell response, specific for the self antigen when applying the inventive compositions, pharmaceutical compositions and vaccines comprising the self antigen to the host. In addition, proteins that result from a combination of two or several self-molecules or that represent a fraction of a self-molecule and proteins that have a high homology two self-molecules as defined above (>95%, preferably >97%, more preferably >99%) may also be considered self.

Specific antibody: As used herein, the term “specific antibody” refers to antibodies which are able to distinguish between the antigen of interest and other antigens and which specifically bind to that antigen of interest with high or low affinity.

High affinity as used herein refers to high attraction between the specific antibody and the antigen of interest, typically and preferably with an affinity constant (Ka) of more than 10⁸ M⁻¹, and thus more than 10⁸ M⁻¹ recognition of free antigen, even more preferably with a Ka of more than 10⁹ M⁻¹, and even more preferably more than 10¹⁰ M⁻¹. The strength of an antibody-antigen interaction depends on both the number of antigen-binding sites occupied and the affinity of each binding site. The strength of the interaction is generally expressed as the affinity constant (Ka). The affinity constant, sometimes called the association constant, can be determined by measuring the concentration of free antigen required to fill half of the antigen-binding sites on the antibody. When half the sites are filled, [AgAb]=[Ab] and Ka=1/[Ag].

Low affinity as used herein refers to low attraction between the specific antibody and the antigen of interest, typically and preferably with Ka of less than 10⁷ M⁻¹, and thus less than 10⁷ M⁻¹ recognition of free antigen, more preferably with a Ka of less than 10⁶ M⁻¹, and even more preferably less than 10⁵ M⁻¹. Methods to measure the affinity of an antibody to an antigen are well known in the art, for example equilibrium dialysis, solid phase methods, affinity chromatography, Biacore®, ELISA (enzyme-linked immunosorbent assay), or any method capable of detecting enzymatic reactions, or RIA (radioimmunoassay).

Treatment: As used herein, the terms “treatment”, “treat”, “treated” or “treating” refer to prophylaxis and/or therapy. When used with respect to an infectious disease, for example, the term refers to a prophylactic treatment which increases the resistance of a subject to infection with a pathogen or, in other words, decreases the likelihood that the subject will become infected with the pathogen, or show signs of illness attributable to the infection, as well as a treatment after the subject has become infected in order to fight the infection, e.g., reduce or eliminate the infection or prevent it from becoming worse.

Virus-like particle (VLP): As used herein, the term “virus-like particle” refers to a structure resembling a virus particle but which has not been demonstrated to be pathogenic. Typically, a virus-like particle in accordance with the invention does not carry genetic information encoding for the proteins of the virus-like particle. In general, virus-like particles lack the viral genome and, therefore, are noninfectious. Also, virus-like particles can often be produced in large quantities by heterologous expression and can be easily purified. Some virus-like particles may contain nucleic acid distinct from their genome. As indicated, a virus-like particle in accordance with the invention is non replicative and noninfectious since it lacks all or part of the viral genome, in particular the replicative and infectious components of the viral genome. A virus-like particle in accordance with the invention may contain nucleic acid distinct from their genome. A typical and preferred embodiment of a virus-like particle in accordance with the present invention is a viral capsid such as the viral capsid of the corresponding virus, bacteriophage, or RNA-phage. The terms “viral capsid” or “capsid”, as interchangeably used herein, refer to a macromolecular assembly composed of viral protein subunits. Typically and preferably, the viral protein subunits assemble into a viral capsid and capsid, respectively, having a structure with an inherent repetitive organization, wherein said structure is, typically, spherical or tubular. For example, the capsids of RNA-phages or HBcAg's have a spherical form of icosahedral symmetry. The term “capsid-like structure” as used herein, refers to a macromolecular assembly composed of viral protein subunits ressembling the capsid morphology in the above defined sense but deviating from the typical symmetrical assembly while maintaining a sufficient degree of order and repetitiveness.

Virus-like particle of a bacteriophage: As used herein, the term “virus-like particle of a bacteriophage” refers to a virus-like particle resembling the structure of a bacteriophage, being non replicative and noninfectious, and lacking at least the gene or genes encoding for the replication machinery of the bacteriophage, and typically also lacking the gene or genes encoding the protein or proteins responsible for viral attachment to or entry into the host. This definition should, however, also encompass virus-like particles of bacteriophages, in which the aforementioned gene or genes are still present but inactive, and, therefore, also leading to non-replicative and noninfectious virus-like particles of a bacteriophage.

VLP of RNA phage coat protein: The capsid structure formed from the self-assembly of 180 subunits of RNA phage coat protein and optionally containing host RNA is referred to as a “VLP of RNA phage coat protein”. A specific example is the VLP of Qβ coat protein. In this particular case, the VLP of Qβ coat protein may either be assembled exclusively from Qβ CP subunits (generated by expression of a Qβ CP gene containing, for example, a TAA stop codon precluding any expression of the longer A1 protein through suppression, see Kozlovska, T. M., et al., Intervirology 39: 9-15 (1996)), or additionally contain A1 protein subunits in the capsid assembly.

Virus particle: The term “virus particle” as used herein refers to the morphological form of a virus. In some virus types it comprises a genome surrounded by a protein capsid; others have additional structures (e.g., envelopes, tails, etc.).

Non-enveloped viral particles are made up of a proteinaceous capsid that surrounds and protects the viral genome. Enveloped viruses also have a capsid structure surrounding the genetic material of the virus but, in addition, have a lipid bilayer envelope that surrounds the capsid. In a preferred embodiment of the invention, the VLP's are free of a lipoprotein envelope or a lipoprotein-containing envelope. In a further preferred embodiment, the VLP's are free of an envelope altogether.

One, a, or an: When the terms “one,” “a,” or “an” are used in this disclosure, they mean “at least one” or “one or more,” unless otherwise indicated.

As will be clear to those skilled in the art, certain embodiments of the invention involve the use of recombinant nucleic acid technologies such as cloning, polymerase chain reaction, the purification of DNA and RNA, the expression of recombinant proteins in prokaryotic and eukaryotic cells, etc. Such methodologies are well known to those skilled in the art and can be conveniently found in published laboratory methods manuals (e.g., Sambrook, J. et al., eds., Molecular Cloning, A Laboratory Manual, 2nd. edition, Cold Spring Harbor Laboratory Press, Cold Spring Harbor, N.Y. (1989); Ausubel, F. et al., eds., Current Protocols in Molecular Biology, John H. Wiley & Sons, Inc. (1997)). Fundamental laboratory techniques for working with tissue culture cell lines (Celis, J., ed., Cell Biology, Academic Press, 2nd edition, (1998)) and antibody-based technologies (Harlow, E. and Lane, D., “Antibodies: A Laboratory Manual,” Cold Spring Harbor Laboratory, Cold Spring Harbor, N.Y. (1988); Deutscher, M. P., “Guide to Protein Purification,” Meth. Enzymol. 128, Academic Press San Diego (1990); Scopes, R. K., “Protein Purification Principles and Practice,” 3rd ed., Springer-Verlag, New York (1994)) are also adequately described in the literature, all of which are incorporated herein by reference.

2. Methods for Detection of Specific B Cells

The disclosed invention provides methods for detection, selection and isolation of specific B cells from animals or humans using compositions comprising, or alternatively consisting of a repetitive core structure such as a virus or a virus-like particle and an antigen coupled, fused or attached otherwise to the core particle. The present invention, thus, provides a method for selecting at least one antigen-specific B cell from a mixture of cells, said method comprising, or alternatively consisting essentially of, or alternatively consisting of: (A) providing a mixture of cells comprising B cells; (B) providing a first composition comprising, or alternatively consisting essentially of, or alternatively consisting of: (a) a first core particle with at least one first attachment site; and (b) at least one antigen or antigenic determinant with at least one second attachment site, wherein said second attachment site being selected from the group consisting of: (i) an attachment site not naturally occurring with said antigen or antigenic determinant; and (ii) an attachment site naturally occurring with said antigen or antigenic determinant, wherein said second attachment site is capable of association to said first attachment site; and wherein said antigen or antigenic determinant and said first core particle interact through said association to form an ordered and repetitive antigen array; (C) contacting said mixture of cells with said first composition; (D) labeling said first composition with a first labeling compound; (E) labeling said B cells in said mixture of cells with a second labeling compound; and (F) selecting at least one B cell which is positive for the first and said second labeling compound.

In a first step of the method of the present invention, a mixture of cells comprising B cells is provided. Any mixture of cells comprising B cells can be used for the method of the present invention, preferably a cell suspension of cells of the peripheral lymphoid organs or of peripheral blood cells. Cells of the peripheral lymphoid organs may include without limitation splenocytes from the spleen or lymphocytes from lymph nodes. Cells from any organ may be used for the method of the present invention which may include without limitation cells from the bone marrow, gut, or lung. Cells used for the method of the present invention are from animals, preferably mammals, even more preferably from mammals immunized with an antigen of interest, preferably immunized with the second composition of the invention. In a preferred aspect of the method of the present invention, said mixture of cells is a mixture of splenocytes from immunized animals, said animals being immunized with a second composition comprising, or alternatively consisting essentially of, or alternatively consisting of: (a) a second core particle with at least one first attachment site; and (b) at least one antigen or antigenic determinant with at least one second attachment site, wherein said second attachment site being selected from the group consisting of: (i) an attachment site not naturally occurring with said antigen or antigenic determinant; and (ii) an attachment site naturally occurring with said antigen or antigenic determinant, wherein said second attachment site is capable of association to said first attachment site; wherein said antigen or antigenic determinant and said second core particle interact through said association to form an ordered and repetitive antigen array. In a preferred embodiment, said second core particle is different from said first core particle. In a further embodiment, said second core particle is the same as said first core particle. In a preferred embodiment, the second composition used for the method of the invention comprises the same antigen or antigenic determinant as the antigen or antigenic determinant of the first composition. Alternatively, the second composition used for the method of the invention comprises a different antigen or antigenic determinant as the antigen or antigenic determinant of the first composition.

In another embodiment of the method of the present invention, said mixture of cells is contacted with at least one first composition of the invention. Typically and preferably, said contacting may include without limitation adding, mixing, incubating, or otherwise bringing the first composition together with said mixture of cells. In one embodiment, said first composition may be added to said mixture of cells. In another embodiment, said mixture of cells may be mixed with said first composition. In yet another embodiment, said mixture of cells may be incubated with said first composition of the invention.

In one aspect of the present invention, said labeling of said first composition is effected prior to contacting said mixture of cells with said first composition. Said first composition may be labeled directly with for example a fluorochrome, a magnetic label or a radioactively labeled tag, or it may be labeled indirectly prior to said incubation, with a labeling compound which may be selected from the group consisting of without limitation avidin or biotin, dioxigenin, flag tag or any other tag, and after said incubation with labeled streptavidin, anti-dioxigenin, anti-flag or any other anti-tag. For one preferred embodiment, said first composition, preferably the first core particle of said first composition, is biotinylated prior to contacting said mixture with said first composition. After said contacting said mixture of cells with said first composition, labeled streptavidin, preferably fluorochrome labeled streptavidin, is added to said mixture of cells which binds to said biotinylated first composition which has specifically bound to B cells which are specific for the antigen or antigenic determinant of said first composition.

In another aspect of the invention, said labeling of said first composition is effected after contacting said mixture with said first composition, preferably with at least one antibody probe, wherein said probe comprises an antibody which is specific for said first composition, preferably specific for said core particle of said first composition, said antibody being conjugated with said first labeling compound. Said first labeling compound may be selected from the group consisting of without limitation fluorescent materials or a first fluorochrome (e.g. texas red, rhodamine, Cy5, phycoerythrin (PE), green fluorescent protein (GFP), a tandem dye (e.g. PE-Cy5), fluorescein isothiocyanate (FITC)), magnetic beads, radiolabel (e.g. 131I-labeled antibody, 90Y (a pure beta emitter)-labeled antibody, 211At-labeled antibody), an enzyme, avidin or biotin, or any other tag or label known in the art useful for labeling the composition of the invention. Typically and preferably, said antibody which is specific for said first composition may be a monoclonal or polyclonal antibody specific for said core particle of said first composition, preferably a polyclonal anti-Qβ, or an anti-HBcAg antibody from any species, preferably from a mammal, more preferably from a human, mouse, rat, rabbit, goat, guinea pig, camel, donkey, horse or chicken.

In another preferred embodiment, said labeling effected after contacting said mixture with said first composition is effected with at least one first antibody probe, wherein said first probe comprises a first antibody, preferably an unlabeled first antibody, which is specific for said first composition, preferably specific for said first core particle of said first composition, and at least one second antibody probe, wherein said second probe comprises a second antibody which is specific for said first antibody, said second antibody being conjugated with said first labeling compound. Typically and preferably, said second antibody which is specific for said first antibody may be a monoclonal or polyclonal antibody conjugated with a labeling compound which recognizes the species of the first antibody and may be selected without limitation from the group consisting of anti-rat, anti-mouse, anti-rabbit, anti-goat, or anti-donkey immunoglobulin antibodies.

In a preferred embodiment, said first antibody is specific for said first core particle. Alternatively, said first antibody is specific for the antigen or antigenic determinant of the first composition. The use of a first antibody specific for the first core particle of the invention makes the method of the invention in particularly useful for the detection and isolation of B cells expressing unknown or unavailable antibodies. Thus, the present invention provides a general method for detecting, selecting and isolating an antigen-specific B cell for the use of the production of any monoclonal antibodies of interest.

In a further aspect of the present invention said labeling said B cells in said mixture of cells is effected with a first set of at least one first targeting molecule, wherein said first targeting molecule is specific for at least one B cell marker, and wherein said first targeting molecule is labeled with said second labeling compound. Said first targeting molecule may be any molecule which is specific for at least one B cell marker, preferably an antibody or an F(ab′)2 molecule specific for IgG conjugated with a second labeling compound. Alternatively, labeling said B cells in said mixture of cells is effected with a first targeting molecule which is a first antibody specific for said at least one B cell marker, and with at least one second antibody probe, wherein said second probe comprises a second antibody which is specific for said first antibody, said second antibody being conjugated with said second labeling compound. Said second labeling compound may be selected from the group consisting of without limitation fluorescent materials or fluorochrome (e.g. texas red, rhodamine, Cy5, phycoerythrin (PE), green fluorescent protein (GFP), a tandem dye (e.g. PE-Cy5), fluorescein isothiocyanate (FITC)), magnetic beads, radiolabel (e.g. 131I-labeled antibody, 90Y (a pure beta emitter)-labeled antibody, 211At-labeled antibody), or any label known in the art useful for labeling at least one B cell marker of the invention. Preferably, said second labeling compound is a second fluorochrome. Even more preferably, said second fluorochrome may yield a color different from that of said first fluorochrome upon activation. A B cell marker can be any surface molecule on B cells which is specific for antigen-specific IgG-producing B cells. A B cell marker can be selected from the group consisting of without limitation the surface IgG, kappa and lambda chains, Ig-alpha (CD79alpha), Ig-beta (CD70beta), CD19, Ia, Fc receptors, B220 (CD45R), CD20, CD21, CD22, CD23, CD81 (TAPA-1), any other CD antigen specific for B cells, or any other marker specific for B cells known in the art. Preferably, said B cell marker is CD19. In another preferred embodiment, said fluorescence conjugated first targeting molecule is a phycoerythrin-conjugated anti-CD19 antibody. In another embodiment, more than one targeting molecule specific for a B cell may be used.

In still a further embodiment, the method of the present invention further comprises labeling said mixture with a second set of at least one second additional targeting molecule, wherein said at least one second targeting molecule is specific for at least one marker unique for cells other than isotype-switched B cells, and wherein said at least one second targeting molecule is labeled with a third labeling compound. Said second targeting molecule may be any molecule which is specific for at least one marker unique for cells other than isotype-switched B cells, preferably an antibody conjugated with a third labeling compound. Said third labeling compound may be selected from the group consisting of without limitation fluorescent material or a third fluorochrome (e.g. texas red, rhodamine, Cy5, phycoerythrin (PE), green fluorescent protein (GFP), a tandem dye (e.g. PE-Cy5), fluorescein isothiocyanate (FITC)), magnetic beads, radiolabel (e.g. 131I-labeled antibody, 90Y (a pure beta emitter)-labeled antibody, 211At-labeled antibody), or any label known in the art useful for labeling at least one B cell marker of the invention. Preferably, said third labeling compound is a third fluorochrome. More preferably, said third fluorochrome may yield a color different from that of said first and said second fluorochrome. Markers for cells other than isotype-switched B cells may be selected from the group consisting of without limitation IgD, IgM, CD2, CD3, CD4, CD8, CD11b, Gr-1, Thy-1, CD43. However, any other markers for isotype-switched B cells known in the art can be used for the method of the present invention. Said second set of at least one second additional targeting molecule may be a set of one or more than one additional targeting molecule. In a preferred embodiment, said second set of additional targeting molecules comprises a mixture of FITC-conjugated antibodies containing anti-IgD, anti-IgM, anti-CD4, anti-CD8, anti-CD11b, and anti-Gr-1 antibodies. The use of more than one different second targeting molecule specific for cells other than isotype-switched B cells is advantageous to efficiently separate isotype-switched B cells from cells other than isotype-switched B cells, and in particular, to minimize background staining. In another embodiment, the present invention may further comprise adding a dead cell marker to said mixture of cells. Dead cell markers are markers which specifically label dead cells. Such markers may be selected from the group consisting of without limitation propidium iodide (PI), YO Pro1 (YO-PRO®-1 iodide (491/509)), 7-AAD (7-aminoactinomycin D), EMA (ethidium monoazide bromide), BIS-Oxonol, To-Pro-3 (see Molecular Probes (Cat No T-3605)), RB2Z.

In a preferred embodiment, the method of the invention comprises the step of selecting at least one B cell which is positive for said first and said second labeling compound. “Positive” B cells means any B cell which is labeled with any one of the labeling compounds of the invention and which is selected or sorted or otherwise separated from a mixture of cells by a device capable of detecting said labeling compound. In a more preferred embodiment, the present invention comprises the step of selecting B cells which are positive for said first and said second labeling compound, but eliminating and not selecting those cells which are positive for said third labeling compound of the invention. In an even more preferred embodiment, the present invention comprises the step of selecting B cells which are positive for said first and said second labeling compound, but eliminating and not selecting those cells which are positive for said third labeling compound of the invention and eliminating and not selecting those cells which are positive for the dead cell marker.

In one embodiment of the present invention, said selecting at least one B cell which is positive for said first and said second labeling compound is effected by using a first device capable of detecting said first labeling compound and a second device capable of detecting said second labeling compound. Any device capable of detecting a labeling compound known in the art may be useful for the method of the invention. A device capable of detecting a fluorochrome may be selected from the group consisting of without limitation a fluorescence activated cell sorting apparatus (FACS) (Dangl and Herzenberg (1982), J. Immunol. Methods 52, 1-14; or Marder et al., (1990), Cytometry 11: 498-505). A device capable of detecting a magnetic particle may include without limitation a magnetic cell sorting apparatus (MACS) (Irsch et al., (1995), Immunotechnology, 1(2):115-25). Typically and preferably, said fluorescence activated cell sorting may be performed with a fluorescence activated cell sorting apparatus (for example FACSVantage or Star Plus™ from Becton Dickinson (Foster City, Calif.), or Epics C from Coulter Epics Division (Hialeah, Fla.)), which can sort individual cells into wells of 8×12 well microculture plates. Any combination of hereinabove mentioned devices capable of detecting a labeling compound of the invention is within the scope of the invention.

In one embodiment of the present invention, said device capable of detecting said first labeling compound is a fluorescence activated cell sorting apparatus (FACS) and said first labeling compound is a first fluorochrome. In another embodiment, said first and said second labeling compounds are fluorochromes, said first fluorochrome yielding a color different from said second fluorochrome upon activation, and said device capable of detecting said first and said second labeling compound is a fluorescence activated cell sorting apparatus (FACS). Alternatively, said first labeling compound is a magnetic label and said device capable of detecting said first labeling compound is a magnetic cell sorting apparatus (MACS), and said second labeling compounds is a fluorochrome and said device capable of detecting said second labeling compound is a fluorescence activated cell sorting apparatus (FACS). In yet another embodiment, said first labeling compound is a first fluorochrome and said device capable of detecting said first labeling compound is a fluorescence activated cell sorting apparatus (FACS), and said second labeling compound is a magnetic label and said device capable of detecting said second labeling compound is a magnetic cell sorting apparatus (MACS).

In one aspect of the invention, said third labeling compound is a third fluorochrome, and the device capable of detecting said third labeling compound is a fluorescence activated cell sorting apparatus (FACS). In one embodiment, said first, said second, and said third labeling compounds of the invention are first, second and third fluorochromes, said third fluorochrome yielding a color different from said first and said second fluorochrome upon activation. In another embodiment, said first and said third labeling compounds of the invention are fluorochromes and said second labeling compound is a magnetic label, said third fluorochrome yielding a color different from said first fluorochrome upon activation. In yet another embodiment, said first labeling compound of the invention is a magnetic label and said second and third labeling compounds are fluorochromes, said third fluorochrome yielding a color different from said second fluorochrome upon activation. In another aspect of the invention, said third labeling compound is a magnetic label and said device capable of detecting said third labeling compound is a magnetic cell sorting apparatus (MACS).

In a preferred embodiment of the invention, said first labeling compound is a first fluorochrome, said second labeling compound is a second fluorochrome, and said third labeling compound is a third fluorochrome, said first, second, and third fluorochromes yielding different colors upon activation, and wherein said device capable of detecting said first, second, and third labeling compound is a fluorescence activated cell sorting apparatus (FACS), preferably a FACSVantage. The usage of a FACSVantage is particularly advantageous for the sorting and isolation of single antigen-specific B cells into separate compartments or wells, and in particular for the subsequent generation of monoclonal antibodies, and no further step of isolating the single antigen-specific B cell is needed. However, in a further embodiment, the method of the invention further comprises the step of isolating at least one antigen-specific B cell which is positive for said first and said second labeling compound.

In one embodiment, the method of the present invention further comprises the step of verifying specific antibody production of said selected at least one antigen-specific B cell. Typically and preferably, the method for verifying the specificity and affinity of the specific antibodies produced by the selected B cells may be selected from the group consisting without limitation of ELISA (enzyme-linked immunosorbent assay), or any method capable of detecting enzymatic reactions, or RIA (radioimmunoassay).

The present invention further provides an antigen-specific B cell selected by any method of the invention. Said antigen-specific B cell may specifically express antibodies that are able to distinguish between the antigen of interest and other antigens and which specifically bind to that antigen of interest with high or low affinity but which do not bind other antigens. Thus said B cells selected by any method of the invention may be used for the production of monoclonal antibodies of interest. Thus, the present invention further provides a method for generating monoclonal antibodies, comprising providing at least one antigen-specific B cell selected by the method of the invention and fusing said at least one antigen-specific B cell with a myeloma cell line.

In a further aspect, the present invention provides a method for generating monoclonal antibodies or antibody fragments comprising isolating at least one genetic element encoding the immunoglobulin or parts of the immunoglobulin expressed by said at least one antigen-specific B cell selected by the method of the invention and expressing said genetic elements. Typically and preferably, said genetic elements of the selected antigen-specific B cell may be selected from the group consisting of without limitation the VH gene segment, the VL gene segment, the VH nucleotide sequence, the VL nucleotide sequence, or any part of the immunoglobulin expressed by the selected antigen-specific B cell. Preferably, RNA is isolated from the selected antigen-specific B cell for the generation of cDNA encoding the variable region of immunoglobulin encoded by said B cell for recombinant production of monoclonal antibodies. In a further embodiment, said genetic elements are amplified by PCR. Techniques such as PCR that can be performed on single B cells to amplify the VH and VL segments are well known in the art (Chiang et al., (1989), Biotechniques, 7(4):360-6).

In yet a further embodiment, said genetic elements may be expressed as a fusion molecule. Alternatively, the selected antigen-specific B cell may be fused in vitro with a fusion partner, allowing the generation of hybridomas secreting monoclonal antibodies of the desired specificity.

Monoclonal antibodies can be obtained by injecting mice with the second composition of the invention comprising a second core particle and at least one antigen, subsequently verifying the presence of antibody production by removing a serum sample, removing the spleen to obtain B-lymphocytes, selecting an antigen-specific B cell by a method of the invention, fusing the selected B-lymphocytes with myeloma cells to produce hybridomas, culturing the clones that produce antibodies to the antigen, and isolating the antibodies from the hybridoma cultures.

Monoclonal antibodies can be isolated and purified from hybridoma cultures by a variety of well-established techniques. Such isolation techniques include affinity chromatography with Protein-A Sepharose, size-exclusion chromatography, and ion-exchange chromatography. See, for example, Coligan at pages 2.7.1-2.7.12 and pages 2.9.1-2.9.3. Also, see Baines et al., “Purification of Immunoglobulin G (IgG),” in METHODS IN MOLECULAR BIOLOGY, VOL. 10, pages 79-104 (The Humana Press, Inc. 1992). Suitable host mammals for the production of antibodies include, but are not limited to, humans, rats, mice, rabbits, and goats.

In accordance with the present invention, functional antibody fragments also can be utilized. The fragments are produced by methods that include digestion with enzymes such as pepsin or papain and/or cleavage of disulfide bonds by chemical reduction. Alternatively, antibody fragments encompassed by the present invention can be synthesized using an automated peptide synthesizer such as those supplied commercially by Applied Biosystems, Multiple Peptide Systems and others, or they may be produced manually, using techniques well known in the art. See Geysen et al., J. Immunol. Methods 102: 259 (1978). Direct determination of the amino acid sequences of the variable regions of the heavy and light chains of the monoclonal antibodies according to the invention can be carried out using conventional techniques.

A fragment according to the present invention can be an Fv fragment. An Fv fragment of an antibody is made up of the variable region of the heavy chain (Vh) of an antibody and the variable region of the light chain of an antibody (V1). Proteolytic cleavage of an antibody can produce double chain Fv fragments in which the Vh and V1 regions remain non-covalently associated and retain antigen binding capacity. Fv fragments also include recombinant single chain antibody molecules in which the light and heavy chain variable regions are connected by a peptide linker. See Skerra, et al. Science, 240, 1038-41 (1988). Antibody fragments according to the invention also include Fab, Fab′, F(ab).sub.2, and F(ab′).sub.2, which lack the Fc fragment of an intact antibody.

Monoclonal antibodies obtained by a method of the invention may be used for research purposes, diagnostic purposes or the treatment of diseases.

3. Compositions Used for the Method of the Invention

In one embodiment of the method of the present invention, a first composition is provided comprising, or alternatively consisting essentially of, or alternatively consisting of: (a) a first core particle with at least one first attachment site; and (b) at least one antigen or antigenic determinant with at least one second attachment site, wherein said second attachment site being selected from the group consisting of: (i) an attachment site not naturally occurring with said antigen or antigenic determinant; and (ii) an attachment site naturally occurring with said antigen or antigenic determinant, wherein said second attachment site is capable of association to said first attachment site; and wherein said antigen or antigenic determinant and said first core particle interact through said association to form an ordered and repetitive antigen array.

In a further aspect of the method of the present invention, a mixture of cells is provided, preferably but not necessarily a mixture of splenocytes from immunized animals, said animals being immunized with a second composition comprising, or alternatively consisting essentially of, or alternatively consisting of: (a) a second core particle with at least one first attachment site; and (b) at least one antigen or antigenic determinant with at least one second attachment site, wherein said second attachment site being selected from the group consisting of: (i) an attachment site not naturally occurring with said antigen or antigenic determinant; and (ii) an attachment site naturally occurring with said antigen or antigenic determinant, wherein said second attachment site is capable of association to said first attachment site; wherein said antigen or antigenic determinant and said second core particle interact through said association to form an ordered and repetitive antigen array. In a preferred embodiment, said second core particle is different from said first core particle. Alternatively, said second core particle is the same as said first core particle. In a preferred embodiment, the second composition used for the method of the invention comprises the same antigen or antigenic determinant as the antigen or antigenic determinant of the first composition. Alternatively, the second composition used for the method of the invention comprises a different antigen or antigenic determinant as the antigen or antigenic determinant of the first composition.

Preferred embodiments of core particles of any one of the compositions suitable for use in the present invention are a virus, a virus-like particle, in particular a Hepatitis B virus core antigen, a bacteriophage, a bacterial pilus or flagella, a viral capsid particle, a virus-like particle of a RNA-phage, in particular a virus-like particle of a RNA-phage Qβ, or any recombinant form of the aforementioned core particles, or any other core particle having an inherent repetitive structure capable of forming an ordered and repetitive antigen array in accordance with the present invention. More specifically, said composition of the invention may comprise, or alternatively consist of, a virus-like particle and at least one antigen or antigenic determinant, wherein the at least one antigen or antigenic determinant is bound to the virus-like particle so as to form an ordered and repetitive antigen-VLP-array. Furthermore, the invention conveniently enables the practitioner to construct such a composition, inter alia, for the use for methods for detection, selection and isolation of antigen-specific B cells.

In one embodiment, the first and/or the second core particle comprises, alternatively essentially consists of, or alternatively consists of a virus, a bacterial pilus, a structure formed from bacterial pilin, a bacteriophage, a virus-like particle, a viral capsid particle or a recombinant form thereof. Any virus, e.g. an RNA or DNA virus, known in the art having an ordered and repetitive coat and/or core protein structure may be selected as a core particle of the invention; examples of suitable viruses include sindbis and other alphaviruses, rhabdoviruses (e.g. vesicular stomatitis virus), picomaviruses (e.g., human rhino virus, Aichi virus), togaviruses (e.g., rubella virus), orthomyxoviruses (e.g., Thogoto virus, Balken virus, fowl plague virus), polyomaviruses (e.g., polyomavirus BK, polyomavirus JC, avian polyomavirus BFDV), parvoviruses, rotaviruses, Norwalk virus, foot and mouth disease virus, a retrovirus, Hepatitis B virus, Tobacco mosaic virus, Flock House Virus, and human Papilomavirus, and preferably a RNA phage, bacteriophage Qβ, bacteriophage R17, bacteriophage M11, bacteriophage MX1, bacteriophage NL95, bacteriophage fr, bacteriophage GA, bacteriophage SP, bacteriophage MS2, bacteriophage f2, bacteriophage PP7, bacteriophage AP205 (for example, see Table 1 in Bachmann, M. F. and Zinkernagel, R. M., Immunol. Today 17:553-558 (1996)).

The first and/or second core particle useful in the present invention has been described in detail in WO01/85208, WO 03/024480, WO 03/024481, WO 02/056905, WO 04/000351, or WO 04/007538, the disclosure of which is incorporated herein by reference in its entirety.

Examples of RNA viruses suitable for use as core particle in the present invention include, but are not limited to, the following: members of the family Reoviridae, including the genus Orthoreovirus (multiple serotypes of both mammalian and avian retroviruses), the genus Orbivirus (Bluetongue virus, Eugenangee virus, Kemerovo virus, African horse sickness virus, and Colorado Tick Fever virus), the genus Rotavirus (human rotavirus, Nebraska calf diarrhea virus, murine rotavirus, simian rotavirus, bovine or ovine rotavirus, avian rotavirus); the family Picornaviridae, including the genus Enterovirus (poliovirus, Coxsackie virus A and B, enteric cytopathic human orphan (ECHO) viruses, hepatitis A, C, D, E and G viruses, Simian enteroviruses, Murine encephalomyelitis (ME) viruses, Poliovirus muris, Bovine enteroviruses, Porcine enteroviruses, the genus Cardiovirus (Encephalomyocarditis virus (EMC), Mengovirus), the genus Rhinovirus (Human rhinoviruses including at least 113 subtypes; other rhinoviruses), the genus Apthovirus (Foot and Mouth disease (FMDV); the family Calciviridae, including Vesicular exanthema of swine virus, San Miguel sea lion virus, Feline picomavirus and Norwalk virus; the family Togaviridae, including the genus Alphavirus (Eastern equine encephalitis virus, Semliki forest virus, Sindbis virus, Chikungunya virus, ONyong-Nyong virus, Ross river virus, Venezuelan equine encephalitis virus, Western equine encephalitis virus), the genus Flavirius (Mosquito borne yellow fever virus, Dengue virus, Japanese encephalitis virus, St. Louis encephalitis virus, Murray Valley encephalitis virus, West Nile virus, Kunjin virus, Central European tick borne virus, Far Eastern tick borne virus, Kyasanur forest virus, Louping III virus, Powassan virus, Omsk hemorrhagic fever virus), the genus Rubivirus (Rubella virus), the genus Pestivirus (Mucosal disease virus, Hog cholera virus, Border disease virus); the family Bunyaviridae, including the genus Bunyvirus (Bunyarnwera and related viruses, California encephalitis group viruses), the genus Phlebovirus (Sandfly fever Sicilian virus, Rift Valley fever virus), the genus Nairovirus (Crimean-Congo hemorrhagic fever virus, Nairobi sheep disease virus), and the genus Uukuvirus (Uukuniemi and related viruses); the family Orthomyxoviridae, including the genus Influenza virus (Influenza virus type A, many human subtypes); Swine influenza virus, and Avian and Equine Influenza viruses; influenza type B (many human subtypes), and influenza type C (possible separate genus); the family paramyxoviridae, including the genus Paramyxovirus (Parainfluenza virus type 1, Sendai virus, Hemadsorption virus, Parainfluenza viruses types 2 to 5, Newcastle Disease Virus, Mumps virus), the genus Morbillivirus (Measles virus, subacute sclerosing panencephalitis virus, distemper virus, Rinderpest virus), the genus Pneumovirus (respiratory syncytial virus (RSV), Bovine respiratory syncytial virus and Pneumonia virus of mice); forest virus, Sindbis virus, Chikungunya virus, ONyong-Nyong virus, Ross river virus, Venezuelan equine encephalitis virus, Western equine encephalitis virus), the genus Flavirius (Mosquito borne yellow fever virus, Dengue virus, Japanese encephalitis virus, St. Louis encephalitis virus, Murray Valley encephalitis virus, West Nile virus, Kunjin virus, Central European tick borne virus, Far Eastern tick borne virus, Kyasanur forest virus, Louping III virus, Powassan virus, Omsk hemorrhagic fever virus), the genus Rubivirus (Rubella virus), the genus Pestivirus (Mucosal disease virus, Hog cholera virus, Border disease virus); the family Bunyaviridae, including the genus Bunyvirus (Bunyamwera and related viruses, California encephalitis group viruses), the genus Phlebovirus (Sandfly fever Sicilian virus, Rift Valley fever virus), the genus Nairovirus (Crimean-Congo hemorrhagic fever virus, Nairobi sheep disease virus), and the genus Uukuvirus (Uukuniemi and related viruses); the family Orthomyxoviridae, including the genus Influenza virus (Influenza virus type A, many human subtypes); Swine influenza virus, and Avian and Equine Influenza viruses; influenza type B (many human subtypes), and influenza type C (possible separate genus); the family paramyxoviridae, including the genus Paramyxovirus (Parainfluenza virus type 1, Sendai virus, Hemadsorption virus, Parainfluenza viruses types 2 to 5, Newcastle Disease Virus, Mumps virus), the genus Morbillivirus (Measles virus, subacute sclerosing panencephalitis virus, distemper virus, Rinderpest virus), the genus Pneumovirus (respiratory syncytial virus (RSV), Bovine respiratory syncytial virus and Pneumonia virus of mice); the family Rhabdoviridae, including the genus Vesiculovirus (VSV), Chandipura virus, Flanders-Hart Park virus), the genus Lyssavirus (Rabies virus), fish Rhabdoviruses and, filoviruses (Marburg virus and Ebola virus); the family Arenaviridae, including Lymphocytic choriomeningitis virus (LCM), Tacaribe virus complex, and Lassa virus; the family Coronoaviridae, including Infectious Bronchitis Virus (IBV), Mouse Hepatitis virus, Human enteric corona virus, and Feline infectious peritonitis (Feline coronavirus).

Illustrative DNA viruses that may be used as core particles include, but are not limited to: the family Poxyiridae, including the genus Orthopoxvirus (Variola major, Variola minor, Monkey pox Vaccinia, Cowpox, Buffalopox, Rabbitpox, Ectromelia), the genus Leporipoxvirus (Myxoma, Fibroma), the genus Avipoxvirus (Fowlpox, other avian poxvirus), the genus Capripoxvirus (sheeppox, goatpox), the genus Suipoxvirus (Swinepox), the genus Parapoxvirus (contagious postular dermatitis virus, pseudocowpox, bovine papular stomatitis virus); the family Iridoviridae (African swine fever virus, Frog viruses 2 and 3, Lymphocystis virus of fish); the family Herpesviridae, including the alpha-Herpesviruses (Herpes Simplex Types 1 and 2, Varicella-Zoster, Equine abortion virus, Equine herpes virus 2 and 3, pseudorabies virus, infectious bovine keratoconjunctivitis virus, infectious bovine rhinotracheitis virus, feline rhinotracheitis virus, infectious laryngotracheitis virus) the Beta-herpesviruses (Human cytomegalovirus and cytomegaloviruses of swine, monkeys and rodents); the gamma-herpesviruses (Epstein-Barr virus (EBV), Marek's disease virus, Herpes saimiri, Herpesvirus ateles, Herpesvirus sylvilagus, guinea pig herpes virus, Lucke tumor virus); the family Adenoviridae, including the genus Mastadenovirus (Human subgroups A, B, C, D and E and ungrouped; simian adenoviruses (at least 23 serotypes), infectious canine hepatitis, and adenoviruses of cattle, pigs, sheep, frogs and many other species, the genus Aviadenovirus (Avian adenoviruses); and non-cultivatable adenoviruses; the family Papoviridae, including the genus Papillomavirus (Human papilloma viruses, bovine papilloma viruses, Shope rabbit papilloma virus, and various pathogenic papilloma viruses of other species), the genus Polyomavirus (polyomavirus, Simian vacuolating agent (SV-40), Rabbit vacuolating agent (RKV), K virus, BK virus, JC virus, and other primate polyoma viruses such as Lymphotrophic papilloma virus); the family Parvoviridae including the genus Adeno-associated viruses, the genus Parvovirus (Feline panleukopenia virus, bovine parvovirus, canine parvovirus, Aleutian mink disease virus, etc.). Finally, DNA viruses may include viruses such as chronic infectious neuropathic agents (CHINA virus).

In other embodiments, a bacterial pilin, a subportion of a bacterial pilin, or a fusion protein which contains either a bacterial pilin or subportion thereof is used to prepare compositions of the invention. Examples of pilin proteins include pilins produced by Escherichia coli (e.g. P pilin of E. coli (GenBank report AF237482), Haemophilus influenzae, Neisseria meningitidis, Neisseria gonorrhoeae, Caulobacter crescentus, Pseudomonas stutzeri, and Pseudomonas aeruginosa Further preferred compositions used for the method of the invention comprising, alternatively consisting essentially of, or alternatively consisting of pili or pilus-like structures as first and/or second core particle are described in WO 01/85208 and WO 02/056905, the disclosure of which are herewith incorporated by reference in its entirety.

In a preferred embodiment, the first and/or second core particle useful in the present invention is a virus-like particle. Virus-like particles (VLPs) in the context of the present application refer to structures resembling a virus particle but which are not pathogenic. In general, virus-like particles lack the viral genome and, therefore, are noninfectious. Also, virus-like particles can be produced in large quantities by heterologous expression and can be easily purified.

In a preferred embodiment, the virus-like particle is a recombinant virus-like particle. The skilled artisan can produce VLPs using recombinant DNA technology and virus coding sequences which are readily available to the public. For example, the coding sequence of a virus envelope or core protein can be engineered for expression in a baculovirus expression vector using a commercially available baculovirus vector, under the regulatory control of a virus promoter, with appropriate modifications of the sequence to allow functional linkage of the coding sequence to the regulatory sequence. The coding sequence of a virus envelope or core protein can also be engineered for expression in a bacterial expression vector, for example.

Examples of VLPs include, but are not limited to, the capsid proteins of Hepatitis B virus, measles virus, Sindbis virus, rotavirus, foot-and-mouth-disease virus, Norwalk virus, the retroviral GAG protein, the retrotransposon Ty protein p1, the surface protein of Hepatitis B virus, human papilloma virus, RNA phages, Ty, fr-phage, GA-phage, AP205-phage, and Qβ-phage.

As will be readily apparent to those skilled in the art, the VLP of the invention is not limited to any specific form. The particle can be synthesized chemically or through a biological process, which can be natural or non-natural. By way of example, this type of embodiment includes a virus-like particle or a recombinant form thereof.

In a more specific embodiment, the VLP can comprise, or alternatively essentially consist of, or alternatively consist of recombinant polypeptides, or fragments thereof, being selected from recombinant polypeptides of Rotavirus, recombinant polypeptides of Norwalk virus, recombinant polypeptides of Alphavirus, recombinant polypeptides of Foot and Mouth Disease virus, recombinant polypeptides of measles virus, recombinant polypeptides of Sindbis virus, recombinant polypeptides of Polyoma virus, recombinant polypeptides of Retrovirus, recombinant polypeptides of Hepatitis B virus (e.g., a HBcAg), recombinant polypeptides of Tobacco mosaic virus, recombinant polypeptides of Flock House Virus, recombinant polypeptides of human Papillomavirus, recombinant polypeptides of bacteriophages, recombinant polypeptides of RNA phages, recombinant polypeptides of Ty, recombinant polypeptides of fr-phage, recombinant polypeptides of GA-phage, recombinant polypeptides of AP205-phage, and recombinant polypeptides of Qβ-phage. The virus-like particle can further comprise, or alternatively essentially consist of, or alternatively consist of, one or more fragments of such polypeptides, as well as variants of such polypeptides. Variants of polypeptides can share, for example, at least 80%, 85%, 90%, 95%, 97%, or 99% identity at the amino acid level with their wild type counterparts.

In a preferred embodiment, the virus-like particle comprises, consists essentially of, or alternatively consists of recombinant proteins, or fragments thereof, of a RNA-phage. Preferably, the RNA-phage is selected from the group consisting of a) bacteriophage Qβ; b) bacteriophage R17; c) bacteriophage fr; d) bacteriophage GA; e) bacteriophage SP; f) bacteriophage MS2; g) bacteriophage M11; h) bacteriophage MX1; i) bacteriophage NL95; j) bacteriophage f2; k) bacteriophage PP7; and 1) bacteriophage AP205.

In another preferred embodiment of the present invention, the virus-like particle comprises, or alternatively consists essentially of, or alternatively consists of recombinant proteins, or fragments thereof, of the RNA-bacteriophage Qβ or of the RNA-bacteriophage fr.

In a further preferred embodiment of the present invention, the recombinant proteins comprise, or alternatively consist essentially of, or alternatively consist of coat proteins of RNA phages.

RNA-phage coat proteins forming capsids or VLP's, or fragments of the bacteriophage coat proteins compatible with self-assembly into a capsid or a VLP, are, therefore, further preferred embodiments of the present invention. Bacteriophage Qβ coat proteins, for example, can be expressed recombinantly in E. coli. Further, upon such expression these proteins spontaneously form capsids. Additionally, these capsids form a structure with an inherent repetitive organization.

Specific preferred examples of bacteriophage coat proteins which can be used to prepare compositions of the invention include the coat proteins of RNA bacteriophages such as bacteriophage Qβ (SEQ ID NO: 1; PIR Database, Accession No. VCBPQβ referring to Qβ CP and SEQ ID NO:2; Accession No. AAA16663 referring to Qβ A1 protein), bacteriophage R17 (PIR Accession No. VCBPR7), bacteriophage fr (SEQ ID NO:3; PIR Accession No. VCBPFR), bacteriophage GA (SEQ ID NO:4; GenBank Accession No. NP-040754), bacteriophage SP (GenBank Accession No. CAA30374 referring to SP CP and Accession No. NP-695026 referring to SP A1 protein), bacteriophage MS2 (PIR Accession No. VCBPM2), bacteriophage M 11 (GenBank Accession No. AACQ6250), bacteriophage MX1 (GenBank Accession No. AAC14699), bacteriophage NL95 (GenBank Accession No. AAC14704), bacteriophage f2 (GenBank Accession No. P03611), bacteriophage PP7 (SEQ ID NO: 5), bacteriophage AP205 (SEQ ID NO: 18). Furthermore, the A1 protein of bacteriophage Qβ or C-terminal truncated forms missing as much as 100, 150 or 180 amino acids from its C-terminus may be incorporated in a capsid assembly of Qβ coat proteins. Generally, the percentage of QβA1 protein relative to Qβ CP in the capsid assembly will be limited, in order to ensure capsid formation. Further specific examples of bacteriophage coat proteins are described in WO 02/056905 on page 45 and 46 incorporated herein by reference. Further preferred virus-like particles of RNA-phages, in particular of Qβ in accordance of this invention are disclosed in WO 02/056905, the disclosure of which is herewith incorporated by reference in its entirety.

In a further preferred embodiment, the virus-like particle comprises, or alternatively consists essentially of, or alternatively consists of recombinant proteins, or fragments thereof, of a RNA-phage, wherein the recombinant proteins comprise, consist essentially of or alternatively consist of mutant coat proteins of a RNA phage, preferably of mutant coat proteins of the RNA phages mentioned above. In another preferred embodiment, the mutant coat proteins of the RNA phage have been modified by removal of at least one lysine residue by way of substitution, or by addition of at least one lysine residue by way of substitution; alternatively, the mutant coat proteins of the RNA phage have been modified by deletion of at least one lysine residue, or by addition of at least one lysine residue by way of insertion.

In another preferred embodiment, the virus-like particle comprises, or alternatively consists essentially of, or alternatively consists of recombinant proteins, or fragments thereof, of the RNA-bacteriophage Qβ, wherein the recombinant proteins comprise, or alternatively consist essentially of, or alternatively consist of coat proteins having an amino acid sequence of SEQ ID NO: 1, or a mixture of coat proteins having amino acid sequences of SEQ ID NO: 1 and of SEQ ID NO: 2 or mutants of SEQ ID NO: 2 and wherein the N-terminal methionine is preferably cleaved.

In a further preferred embodiment, the virus-like particle comprises, consists essentially of or alternatively consists of recombinant proteins of Qβ, or fragments thereof, wherein the recombinant proteins comprise, or alternatively consist essentially of, or alternatively consist of mutant Qβ coat proteins. In another preferred embodiment, these mutant coat proteins have been modified by removal of at least one lysine residue by way of substitution, or by addition of at least one lysine residue by way of substitution. Alternatively, these mutant coat proteins have been modified by deletion of at least one lysine residue, or by addition of at least one lysine residue by way of insertion.

Four lysine residues are exposed on the surface of the capsid of Qβ coat protein. Qβ mutants, for which exposed lysine residues are replaced by arginines can also be used for the present invention. The following Qβ coat protein mutants and mutant Qβ VLP's can, thus, be used in the practice of the invention: “Qβ-240” (Lys13-Arg; SEQ ID NO:6), “Qβ-243” (Asn 10-Lys; SEQ ID NO:7), “Qβ-250” (Lys 2-Arg, Lys13-Arg; SEQ ID NO:8), “Qβ-251” (SEQ ID NO:9) and “Qβ-259” (Lys 2-Arg, Lys16-Arg; SEQ ID NO:10). Thus, in further preferred embodiment of the present invention, the virus-like particle comprises, consists essentially of or alternatively consists of recombinant proteins of mutant Qβ coat proteins, which comprise proteins having an amino acid sequence selected from the group of a) the amino acid sequence of SEQ ID NO:6; b) the amino acid sequence of SEQ ID NO:7; c) the amino acid sequence of SEQ ID NO:8; d) the amino acid sequence of SEQ ID NO:9; and e) the amino acid sequence of SEQ ID NO: 10. The construction, expression and purification of the above indicated Qβ coat proteins, mutant Q, coat protein VLP's and capsids, respectively, are disclosed in WO02/056905. In particular is hereby referred to Example 18 of above mentioned application.

In a further preferred embodiment of the present invention, the virus-like particle comprises, or alternatively consists essentially of, or alternatively consists of recombinant proteins of Qβ, or fragments thereof, wherein the recombinant proteins comprise, consist essentially of or alternatively consist of a mixture of either one of the foregoing Qβ mutants and the corresponding A1 protein.

In a further preferred embodiment, the virus-like particle comprises, or alternatively essentially consists of, or alternatively consists of recombinant proteins, or fragments thereof, of RNA-phage AP205.

The AP205 genome consists of a maturation protein, a coat protein, a replicase and two open reading frames not present in related phages; a lysis gene and an open reading frame playing a role in the translation of the maturation gene (Klovins, J., et al., J. Gen. Virol. 83: 1523-33 (2002)). AP205 coat protein can be expressed from plasmid pAP283-58 (SEQ ID NO: 79), which is a derivative of pQb10 (Kozlovska, T. M. et al., Gene 137:133-37 (1993)), and which contains an AP205 ribosomal binding site. Alternatively, AP205 coat protein may be cloned into pQb185, downstream of the ribosomal binding site present in the vector. Both approaches lead to expression of the protein and formation of capsids. Vectors pQb10 and pQb185 are vectors derived from pGEM vector, and expression of the cloned genes in these vectors is controlled by the trp promoter (Kozlovska, T. M. et al., Gene 137:133-37 (1993)). Plasmid pAP283-58 (SEQ ID NO:16) comprises a putative AP205 ribosomal binding site in the following sequence, which is downstream of the XbaI site, and immediately upstream of the ATG start codon of the AP205 coat protein: tctagaATTTTCTGCGCACCCATCCCGGGTGGCGCCCAAAGT GAGGAAAATCACatg (bases 77-133 of SEQ ID NO: 16). The vector pQbl85 comprises a Shine Delagarno sequence downstream from the XbaI site and upstream of the start codon (tctagaTTAACCCAACGCGTAGGAG TCAGGCCatg, (SEQ ID NO: 17), Shine Delagarno sequence underlined).

In a further preferred embodiment of the present invention, the virus-like particle comprises, or alternatively essentially consists of, or alternatively consists of recombinant coat proteins, or fragments thereof, of the RNA-phage AP205.

This preferred embodiment of the present invention, thus, comprises AP205 coat proteins that form capsids. Such proteins are recombinantly expressed, or prepared from natural sources. AP205 coat proteins produced in bacteria spontaneously form capsids, as evidenced by Electron Microscopy (EM) and immunodiffusion. The structural properties of the capsid formed by the AP205 coat protein (SEQ ID NO: 18) and those formed by the coat protein of the AP205 RNA phage are nearly indistinguishable when seen in EM. AP205 VLPs are highly immunogenic, and can be linked with antigens and/or antigenic determinants to generate constructs displaying the antigens and/or antigenic determinants oriented in a repetitive manner. High titers are elicited against the so displayed antigens showing that bound antigens and/or antigenic determinants are accessible for interacting with antibody molecules and are immunogenic.

In a further preferred embodiment of the present invention, the virus-like particle comprises, or alternatively essentially consists of, or alternatively consists of recombinant mutant coat proteins, or fragments thereof, of the RNA-phage AP205.

Assembly-competent mutant forms of AP205 VLPs, including AP205 coat protein with the substitution of proline at amino acid 5 to threonine (SEQ ID NO: 19), may also be used in the practice of the invention and leads to a further preferred embodiment of the invention. These VLPs, AP205 VLPs derived from natural sources, or AP205 viral particles, may be bound to antigens to produce ordered repetitive arrays of the antigens in accordance with the present invention.

AP205 P5-T mutant coat protein can be expressed from plasmid pAP281-32 (SEQ ID No. 20), which is derived directly from pQb185, and which contains the mutant AP205 coat protein gene instead of the Qβ coat protein gene. Vectors for expression of the AP205 coat protein are transfected into E. coli for expression of the AP205 coat protein.

Methods for expression of the coat protein and the mutant coat protein, respectively, leading to self-assembly into VLPs, as well as the coupling of antigens or antigenic determinants to those preferred VLPs, are described in Examples 2-11 of the copending U.S. provisional application 60/396,126 which is incorporated herewith by reference in its entirety. Suitable E. coli strains include, but are not limited to, E. coli K802, JM 109, RR1. Suitable vectors and strains and combinations thereof can be identified by testing expression of the coat protein and mutant coat protein, respectively, by SDS-PAGE and capsid formation and assembly by optionally first purifying the capsids by gel filtration and subsequently testing them in an immunodiffusion assay (Ouchterlony test) or Electron Microscopy (Kozlovska, T. M., et al., Gene 137:133-37 (1993)).

AP205 coat proteins expressed from the vectors pAP283-58 and pAP281-32 may be devoid of the initial Methionine amino-acid, due to processing in the cytoplasm of E. coli. Cleaved, uncleaved forms of AP205 VLP, or mixtures thereof are further preferred embodiments of the invention.

In a further preferred embodiment of the present invention, the virus-like particle comprises, or alternatively essentially consists of, or alternatively consists of a mixture of recombinant coat proteins, or fragments thereof, of the RNA-phage AP205 and of recombinant mutant coat proteins, or fragments thereof, of the RNA-phage AP205.

In a further preferred embodiment of the present invention, the virus-like particle comprises, or alternatively essentially consists of, or alternatively consists of fragments of recombinant coat proteins or recombinant mutant coat proteins of the RNA-phage AP205.

Recombinant AP205 coat protein fragments capable of assembling into a VLP and a capsid, respectively are also useful in the practice of the invention. These fragments may be generated by deletion, either internally or at the termini of the coat protein and mutant coat protein, respectively. Insertions in the coat protein and mutant coat protein sequence or fusions of antigen sequences to the coat protein and mutant coat protein sequence, and compatible with assembly into a VLP, are further embodiments of the invention and lead to chimeric AP205 coat proteins, and particles, respectively. The outcome of insertions, deletions and fusions to the coat protein sequence and whether it is compatible with assembly into a VLP can be determined by electron microscopy.

The particles formed by the AP205 coat protein, coat protein fragments and chimeric coat proteins described above, can be isolated in pure form by a combination of fractionation steps by precipitation and of purification steps by gel filtration using e.g. Sepharose CL-4B, Sepharose CL-2B, Sepharose CL-6B columns and combinations thereof. Other methods of isolating virus-like particles are known in the art, and may be used to isolate the virus-like particles (VLPs) of bacteriophage AP205. For example, the use of ultracentrifugation to isolate VLPs of the yeast retrotransposon Ty is described in U.S. Pat. No. 4,918,166, which is incorporated by reference herein in its entirety.

The crystal structure of several RNA bacteriophages has been determined (Golmohammadi, R. et al., Structure 4:543-554 (1996)). Using such information, surface exposed residues can be identified and, thus, RNA-phage coat proteins can be modified such that one or more reactive amino acid residues can be inserted by way of insertion or substitution. As a consequence, those modified forms of bacteriophage coat proteins can also be used for the present invention. Thus, variants of proteins which form capsids or capsid like structures (e.g., coat proteins of bacteriophage Qβ, bacteriophage R17, bacteriophage fr, bacteriophage GA, bacteriophage SP, and bacteriophage MS2) can also be used to prepare compositions of the present invention. Further possible examples of modified RNA bacteriophages as well as variants of proteins and N- and C terminal truncation mutants which form capsids or capsid like structures, as well as methods for preparing such compositions and vaccine compositions, respectively, which are suitable for use in the present invention are described in WO 02/056905 on page 50, line 33 to page 52, line 29, the disclosure of which is herewith incorporated by reference in its entirety.

The invention thus includes compositions prepared from proteins which form capsids or VLP's, methods for preparing these compositions from individual protein subunits and VLP's or capsids, methods for preparing these individual protein subunits, nucleic acid molecules which encode these subunits, and methods for vaccinating and/or eliciting immunological responses in individuals using these compositions of the present invention.

As previously stated, the invention includes virus-like particles or recombinant forms thereof. In one further preferred embodiment, the particles used in compositions of the invention are composed of a Hepatitis B core protein (HBcAg) or a fragment of a HBcAg. In a further embodiment, the particles used in compositions of the invention are composed of a Hepatitis B core protein (HBcAg) or a fragment of a HBcAg protein, which has been modified to either eliminate or reduce the number of free cysteine residues. Thou et al. (J. Virol. 66:5393 5398 (1992)) demonstrated that HBcAgs which have been modified to remove the naturally resident cysteine residues retain the ability to associate and form capsids. Thus, VLP's suitable for use in compositions of the invention include those comprising modified HBcAgs, or fragments thereof, in which one or more of the naturally resident cysteine residues have been either deleted or substituted with another amino acid residue (e.g., a serine residue).

The HBcAg is a protein generated by the processing of a Hepatitis B core antigen precursor protein. A number of isotypes of the HBcAg have been identified and their amino acids sequences are readily available to those skilled in the art. In most instances, compositions of the invention will be prepared using the processed form of a HBcAg (i.e., a HBcAg from which the N terminal leader sequence of the Hepatitis B core antigen precursor protein have been removed).

Further, when HBcAgs are produced under conditions where processing will not occur, the HBcAgs will generally be expressed in “processed” form. For example, when an E. coli expression system directing expression of the protein to the cytoplasm is used to produce HBcAgs of the invention, these proteins will generally be expressed such that the N terminal leader sequence of the Hepatitis B core antigen precursor protein is not present.

The preparation of Hepatitis B virus-like particles, which can be used for the present invention, is disclosed, for example, in WO 00/32227, and hereby in particular in Examples 17 to 19 and 21 to 24, as well as in WO 01/85208, and hereby in particular in Examples 17 to 19, 21 to 24, 31 and 41, and in WO 02/056905. For the latter application, it is in particular referred to Example 23, 24, 31 and 51. All three documents are explicitly incorporated herein by reference.

The present invention also includes HBcAg variants which have been modified to delete or substitute one or more additional cysteine residues. It is known in the art that free cysteine residues can be involved in a number of chemical side reactions. These side reactions include disulfide exchanges, reaction with chemical substances or metabolites that are, for example, injected or formed in a combination therapy with other substances, or direct oxidation and reaction with nucleotides upon exposure to UV light. Toxic adducts could thus be generated, especially considering the fact that HBcAgs have a strong tendency to bind nucleic acids. The toxic adducts would thus be distributed between a multiplicity of species, which individually may each be present at low concentration, but reach toxic levels when together.

In view of the above, one advantage to the use of HBcAgs in compositions which have been modified to remove naturally resident cysteine residues is that sites to which toxic species can bind when antigens or antigenic determinants are attached would be reduced in number or eliminated altogether.

A number of naturally occurring HBcAg variants suitable for use in the practice of the present invention have been identified. Yuan et al., (J. Virol. 73:10122 10128 (1999)), for example, describe variants in which the isoleucine residue at position corresponding to position 97 in SEQ ID NO: 11 is replaced with either a leucine residue or a phenylalanine residue. The amino acid sequences of a number of HBcAg variants, as well as several Hepatitis B core antigen precursor variants, are disclosed in GenBank reports AAF121240, AF121239, X85297, X02496, X85305, X85303, AF151735, X85259, X85286, X85260, X85317, X85298, AF043593, M20706, X85295, X80925, X85284, X85275, X72702, X85291, X65258, X85302, M32138, X85293, X85315, U95551, X85256, X85316, X85296, AB033559, X59795, X85299, X85307, X65257, X85311, X85301 (SEQ ID NO: 12), X85314, X85287, X85272, X85319, AB010289, X85285, AB010289, AF121242, M90520 (SEQ ID NO:13), P03153, AF110999, and M95589, the disclosures of each of which are incorporated herein by reference. The sequences of the hereinabove mentioned Hepatitis B core antigen precursor variants are further disclosed in WO 01/85208 in SEQ ID NOs: 89-138 of the application WO 01/85208. These HBcAg variants differ in amino acid sequence at a number of positions, including amino acid residues which corresponds to the amino acid residues located at positions 12, 13, 21, 22, 24, 29, 32, 33, 35, 38, 40, 42, 44, 45, 49, 51, 57, 58, 59, 64, 66, 67, 69, 74, 77, 80, 81, 87, 92, 93, 97, 98, 100, 103, 105, 106, 109, 113, 116, 121, 126, 130, 133, 135, 141, 147, 149, 157, 176, 178, 182 and 183 in SEQ ID NO:14. Further HBcAg variants suitable for use in the compositions of the invention, and which may be further modified according to the disclosure of this specification are described in WO 01/98333, WO 00/177158 and WO 00/214478.

As noted above, generally processed HBcAgs (i.e., those which lack leader sequences) will be used in the compositions of the invention. The present invention includes compositions which comprise the above described variant HBcAgs.

Whether the amino acid sequence of a polypeptide has an amino acid sequence that is at least 80%, 85%, 90%, 95%, 97% or 99% identical to one of the above wild-type amino acid sequences, or a subportion thereof, can be determined conventionally using known computer programs such the Bestfit program. When using Bestfit or any other sequence alignment program to determine whether a particular sequence is, for instance, 95% identical to a reference amino acid sequence, the parameters are set such that the percentage of identity is calculated over the full length of the reference amino acid sequence and that gaps in homology of up to 5% of the total number of amino acid residues in the reference sequence are allowed.

The amino acid sequences of the hereinabove mentioned HBcAg variants and precursors are relatively similar to each other. Thus, reference to an amino acid residue of a HBcAg variant located at a position which corresponds to a particular position in SEQ ID NO:14, refers to the amino acid residue which is present at that position in the amino acid sequence shown in SEQ ID NO:14. The homology between these HBcAg variants is for the most part high enough among Hepatitis B viruses that infect mammals so that one skilled in the art would have little difficulty reviewing both the amino acid sequence shown in SEQ ID NO:14 and that of a particular BBcAg variant and identifying “corresponding” amino acid residues. Furthermore, the HBcAg amino acid sequence shown in SEQ ID NO:13, which shows the amino acid sequence of a HBcAg derived from a virus which infect woodchucks, has enough homology to the HBcAg having the amino acid sequence shown in SEQ ID NO: 14 that it is readily apparent that a three amino acid residue insert is present in SEQ ID NO:12 between amino acid residues 155 and 156 of SEQ ID NO:14.

The invention also includes compositions which comprise HBcAg variants of Hepatitis B viruses which infect birds, as wells as compositions which comprise fragments of these HBcAg variants. For these HBcAg variants one, two, three or more of the cysteine residues naturally present in these polypeptides could be either substituted with another amino acid residue or deleted prior to their inclusion in compositions of the invention.

As discussed above, the elimination of free cysteine residues reduces the number of sites where toxic components can bind to the HBcAg, and also eliminates sites where cross linking of lysine and cysteine residues of the same or of neighboring HBcAg molecules can occur. Therefore, in another embodiment of the present invention, one or more cysteine residues of the Hepatitis B virus capsid protein have been either deleted or substituted with another amino acid residue. In other embodiments, compositions of the invention will contain HBcAgs from which the C terminal region (e.g., amino acid residues 145 185 or 150 185 of SEQ ID NO: 14) has been removed. Thus, additional modified HBcAgs suitable for use in the practice of the present invention include C terminal truncation mutants. Suitable truncation mutants include HBcAgs where 1, 5, 10, 15, 20, 25, 30, 34, 35, amino acids have been removed from the C terminus.

HBcAgs suitable for use in the practice of the present invention also include N terminal truncation mutants. Suitable truncation mutants include modified HBcAgs where 1, 2, 5, 7, 9, 10, 12, 14, 15, or 17 amino acids have been removed from the N terminus.

Further HBcAgs suitable for use in the practice of the present invention include N and C terminal truncation mutants. Suitable truncation mutants include HBcAgs where 1, 2, 5, 7, 9, 10, 12, 14, 15, and 17 amino acids have been removed from the N terminus and 1, 5, 10, 15, 20, 25, 30, 34, 35 amino acids have been removed from the C terminus.

The invention further includes compositions comprising HBcAg polypeptides comprising, or alternatively essentially consisting of, or alternatively consisting of, amino acid sequences which are at least 80%, 85%, 90%, 95%, 97%, or 99% identical to the above described truncation mutants.

In certain embodiments of the invention, a lysine residue is introduced into a HBcAg polypeptide, to mediate the binding of the antigen or antigenic determinant to the VLP of HBcAg. In preferred embodiments, compositions of the invention are prepared using a HBcAg comprising, or alternatively consisting of, amino acids 1-144, or 1-149, or 1-185 of SEQ ID NO:14, which is modified so that the amino acids corresponding to positions 79 and 80 are replaced with a peptide having the amino acid sequence of Gly-Gly-Lys-Gly-Gly (SEQ ID NO:60), resulting in the BBcAg variant having the amino acid sequence of SEQ ID NO: 15. In further preferred embodiments, the cysteine residues at positions 48 and 107 of SEQ ID NO:14 are mutated to serine (SEQ ID NO: 61). The invention further includes compositions comprising the corresponding polypeptides having amino acid sequences shown in any of the hereinabove mentioned Hepatitis B core antigen precursor variants, which also have above noted amino acid alterations. Further included within the scope of the invention are additional HBcAg variants which are capable of associating to form a capsid or VLP and have the above noted amino acid alterations. Thus, the invention further includes compositions comprising HBcAg polypeptides which comprise, or alternatively consist of, amino acid sequences which are at least 80%, 85%, 90%, 95%, 97% or 99% identical to any of the wild-type amino acid sequences, and forms of these proteins which have been processed, where appropriate, to remove the N terminal leader sequence and modified with above noted alterations.

Compositions of the invention may comprise mixtures of different HBcAgs. Thus, these compositions may be composed of HBcAgs which differ in amino acid sequence. For example, compositions could be prepared comprising a “wild type” HBcAg and a modified HBcAg in which one or more amino acid residues have been altered (e.g., deleted, inserted or substituted).

In a further preferred embodiment of the present invention, the at least one antigen or antigenic determinant is bound to said first and/or second core particle and virus-like particle, respectively, by at least one covalent bond. Preferably, the least one antigen or antigenic determinant is bound to the core particle and virus-like particle, respectively, by at least one covalent bond, said covalent bond being a non-peptide bond leading to a core particle-antigen ordered and repetitive array and a antigen-VLP-array or -conjugate, respectively. This antigen-VLP array and conjugate, respectively, has typically and preferably a repetitive and ordered structure since the at least one, but usually more than one, antigen or antigenic determinant is bound to the VLP in an oriented manner. Preferably, more than 10, 20, 40, 80, 120 antigens or antigenic determinants or proteins are bound to the VLP. The formation of a repetitive and ordered antigen-VLP array and conjugate, respectively, is ensured by an oriented and directed as well as defined binding and attachment, respectively, of the at least one antigen or antigenic determinant to the VLP as will become apparent in the following. Furthermore, the typical inherent highly repetitive and organized structure of the VLP's advantageously contributes to the display of the antigen or antigenic determinant in a highly ordered and repetitive fashion leading to a highly organized and repetitive antigen-VLP array and conjugate, respectively.

Preferably, the antigen or antigenic determinant is bound to the core particle and VLP, respectively, by way of chemical cross-linking, typically and preferably by using a heterobifunctional cross-linker. In preferred embodiments, the hetero-bifunctional crosslinker contains a functional group which can react with preferred first attachment sites, i.e. with the side-chain amino group of lysine residues of the core particle and the VLP or at least one VLP subunit, respectively, and a further functional group which can react with a preferred second attachment site, i.e. a cysteine residue naturally present, made available for reaction by reduction, or engineered on the antigen or antigenic determinant, and optionally also made available for reaction by reduction. Methods of binding of antigen or antigenic determinant to core particles and VLPs, respectively, are disclosed in WO 00/32227, in WO 01/85208, and in WO 02/056905, the disclosures of which are herewith incorporated by reference in its entirety. The first step of the procedure, typically called the derivatization, is the reaction of the core particle or the VLP with the cross-linker. The product of this reaction is an activated core particle or activated VLP, also called activated carrier. In the second step, unreacted cross-linker is removed using usual methods such as gel filtration or dialysis. In the third step, the antigen or antigenic determinant is reacted with the activated carrier, and this step is typically called the coupling step. Unreacted antigen or antigenic determinant may be optionally removed in a fourth step, for example by dialysis. Several hetero-bifunctional cross-linkers are known to the art. These include the preferred cross-linkers SMPH (Pierce), Sulfo-MBS, Sulfo-EMCS, Sulfo-GMBS, Sulfo-SIAB, Sulfo-SMPB, Sulfo-SMCC, SVSB, SIA and other cross-linkers available for example from the Pierce Chemical Company (Rockford, Ill., USA), and having one functional group reactive towards amino groups and one functional group reactive towards cysteine residues. The above mentioned cross-linkers all lead to formation of a thioether linkage. Another class of cross-linkers suitable in the practice of the invention is characterized by the introduction of a disulfide linkage between the antigen or antigenic determinant and the core particle or VLP upon coupling. Preferred cross-linkers belonging to this class include for example SPDP and Sulfo-LC-SPDP (Pierce). The extent of derivatization of the core particle and VLP, respectively, with cross-linker can be influenced by varying experimental conditions such as the concentration of each of the reaction partners, the excess of one reagent over the other, the pH, the temperature and the ionic strength. The degree of coupling, i.e. the amount of antigen or antigenic determinants per subunits of the core particle and VLP, respectively, can be adjusted by varying the experimental conditions described above to match the requirements of the method of the invention. Solubility of the antigen or antigenic determinant may impose a limitation on the amount of antigen or antigenic determinant that can be coupled on each subunit, and in those cases where the obtained composition would be insoluble, where reducing the amount of antigen or antigenic determinants per subunit is beneficial, preferably for the specific detection and isolation of high affinity B cells, and thus for the selection of B cells which express antibodies which bind antigens with high affinity. In a preferred embodiment of the invention, the antigen or antigenic determinant may be coupled, fused, or otherwise attached to the core particle at high density for efficient isolation of all antigen-specific B cells. High density refers to high amounts of antigen presented on the surface of a core particle which can be measured by SDS gel electrophoresis. A method for high density coupling is described in Example 4. In another preferred embodiment of the invention, the antigen or antigenic determinant may be coupled, fused, or otherwise attached to the core particle at low density for specific isolation of high affinity B cells which are B cells having high binding strength for its antigen. Low density refers to low amounts of antigen presented on the surface of a core particle which can be measured by SDS gel electrophoresis. A method for low density coupling is described in Example 4.

A particularly favored method of binding of antigens or antigenic determinants to the core particle and the VLP, respectively, is the linking of a lysine residue on the surface of the core particle and the VLP, respectively, with a cysteine residue on the antigen or antigenic determinant. Thus, in a preferred embodiment of the present invention, the first attachment site is a lysine residue and the second attachment site is a cysteine residue. In some embodiments, engineering of an amino acid linker containing a cysteine residue, as a second attachment site or as a part thereof, to the antigen or antigenic determinant for coupling to the core particle and VLP, respectively, may be required. Alternatively, a cysteine may be introduced either by insertion or mutation within the antigen or antigenic determinant. Alternatively, the cysteine residue may be introduced by chemical coupling.

In general, flexible amino acid linkers are favored. Examples of the amino acid linker are selected from the group consisting of: (a) CGG; (b) N-terminal gamma 1-linker; (c) N-terminal gamma 3-linker; (d) Ig hinge regions; (e) N-terminal glycine linkers; (f) (G)kC(G)n with n=0-12 and k=0-5; (g) N-terminal glycine-serine linkers; (h) (G)kC(G)m(S)l(GGGGS)ₙ with n=0-3, k=0-5, m=0-10, l=0-2 (SEQ ID NO: 21); (i) GGC; (k) GGC-NH2; (1) C-terminal gamma 1-linker; (m) C-terminal gamma 3-linker; (n) C-terminal glycine linkers; (o) (G)nC(G)k with n=0-12 and k=0-5; (p) C-terminal glycine-serine linkers; (q) (G)ₘ(S)l(GGGGS)n(G)oC(G)ₖ with n=0-3, k=0-5, m=0-10, l=0-2, and o=0-8 (SEQ ID NO: 22).

Further preferred examples of amino acid linkers are the hinge region of Immunoglobuins, glycine serine linkers (GGGGS)n (SEQ ID NO: 23), and glycine linkers (G)n all further containing a cysteine residue as second attachment site and optionally further glycine residues. Typically preferred examples of said amino acid linkers are N-terminal gamma1: CGDKTHTSPP (SEQ ID NO: 24); C-terminal gamma 1: DKTHFSPPCG (SEQ ID NO: 25); N-terminal gamma 3: CGGPKPSTPPGSSGGAP (SEQ ID NO: 26); C-terminal gamma 3: PKPSTPPGSSGGAPGGCG (SEQ ID NO: 27); N-terminal glycine linker: GCGGGG (SEQ ID NO: 28); C-terminal glycine linker: OGGGCG (SEQ ID NO: 29); C-terminal glycine-lysine linker: GGKKGC (SEQ ID NO: 30); N-terminal glycine-lysine linker: CGKKGG (SEQ ID NO: 31).

In a further preferred embodiment of the present invention, GGCG (SEQ ID NO: 32), GGC or GGC-NH2 (“NH2” stands for amidation) linkers at the C-terminus of the peptide or CGG at its N-terminus are preferred as amino acid linkers. In general, glycine residues will be inserted between bulky amino acids and the cysteine to be used as second attachment site, to avoid potential steric hindrance of the bulkier amino acid in the coupling reaction.

Other methods of binding the antigen or antigenic determinant to the core particle and the VLP, respectively, include methods wherein the antigen or antigenic determinant is cross-linked to the core particle and the VLP, respectively, using the carbodiimide EDC, and NHS. Further examples hereto are disclosed in WO 02/056905, the disclosures of which is herewith incorporated by reference in its entirety. The antigen or antigenic determinant may also be first thiolated through reaction, for example with SATA, SATP or iminothiolane. The antigen or antigenic determinant, after deprotection if required, may then be coupled to the core particle and the VLP, respectively, as follows. After separation of the excess thiolation reagent, the antigen or antigenic determinant is reacted with the core particle and the VLP, respectively, previously activated with a hetero-bifunctional cross-linker comprising a cysteine reactive moiety, and therefore displaying at least one or several functional groups reactive towards cysteine residues, to which the thiolated antigen or antigenic determinant can react, such as described above. Optionally, low amounts of a reducing agent are included in the reaction mixture. In further methods, the antigen or antigenic determinant is attached to the core particle and the VLP, respectively, using a homo-bifunctional cross-linker such as glutaraldehyde, DSG, BM[PEO]4, BS3, (Pierce Chemical Company, Rockford, Ill., USA) or other known homo-bifunctional cross-linkers with functional groups reactive towards amine groups or carboxyl groups of the core particle and the VLP, respectively.

In a further embodiment, the antigen or antigenic determinant is bound to the core particle and the VLP, respectively, through modification of the carbohydrate moieties present on glycosylated antigen or antigenic determinant and subsequent reaction with the core particle and the VLP, respectively. Preferred examples of this type of binding are described in WO 02/056905, the disclosures of which is herewith incorporated by reference in its entirety.

Other methods of binding the VLP to a antigen or antigenic determinant include methods where the core particle and the VLP, respectively, is biotinylated, and the antigen or antigenic determinant expressed as a streptavidin-fusion protein, or methods wherein both the antigen or antigenic determinants and the core particle and the VLP, respectively, are biotinylated, for example as described in WO 00/23955. In this case, the antigen or antigenic determinant may be first bound to streptavidin or avidin by adjusting the ratio of antigen or antigenic determinant to streptavidin such that free binding sites are still available for binding of the core particle and the VLP, respectively, which is added in the next step. Alternatively, all components may be mixed in a “one pot” reaction. Other ligand-receptor pairs, where a soluble form of the receptor and of the ligand is available, and are capable of being cross-linked to the core particle and the VLP, respectively, or the antigen or antigenic determinant, may be used as binding agents for binding the antigen or antigenic determinant to the core particle and the VLP, respectively. Alternatively, either the ligand or the receptor may be fused to the antigen or antigenic determinant and so mediate binding to the core particle and the VLP, respectively, chemically bound or fused either to the receptor, or the ligand respectively. Fusion may also be effected by insertion or substitution.

One or several antigen molecules, can be attached to one subunit of the capsid or VLP of RNA phages coat proteins, preferably through the exposed lysine residues of the VLP of RNA phages, if sterically allowable. A specific feature of the VLP of the coat protein of RNA phages and in particular of the Qβ coat protein VLP is thus the possibility to couple several antigens per subunit This allows for the generation of a dense antigen array.

In a preferred embodiment of the invention, the binding and attachment, respectively, of the at least one antigen or antigenic determinant to the core particle and the virus-like particle, respectively, is by way of interaction and association, respectively, between at least one first attachment site of the virus-like particle and at least one second attachment of the antigen or antigenic determinant.

VLPs or capsids of Qβ coat protein display a defined number of lysine residues on their surface, with a defined topology with three lysine residues pointing towards the interior of the capsid and interacting with the RNA, and four other lysine residues exposed to the exterior of the capsid. These defined properties favor the attachment of antigens to the exterior of the particle, rather than to the interior of the particle where the lysine residues interact with RNA. VLPs of other RNA phage coat proteins also have a defined number of lysine residues on their surface and a defined topology of these lysine residues.

In further preferred embodiments of the present invention, the first attachment site is a lysine residue and/or the second attachment comprises sulfhydryl group or a cysteine residue. In a very preferred embodiment of the present invention, the first attachment site is a lysine residue and the second attachment is a cysteine residue.

In very preferred embodiments of the invention, the antigen or antigenic determinant is bound via a cysteine residue, either naturally present on the antigen or antigenic determinant or engineered, to lysine residues of the VLP of RNA phage coat protein, and in particular to the VLP of Qβ coat protein.

Another advantage of the VLPs derived from RNA phages is their high expression yield in bacteria that allows production of large quantities of material at affordable cost.

As indicated, the inventive conjugates and arrays, respectively, differ from prior art conjugates in their highly organized structure, dimensions, and in the repetitiveness of the antigen on the surface of the array. Moreover, the use of the VLPs as carriers allow the formation of robust antigen arrays and conjugates, respectively, with variable antigen density. In particular, the use of VLP's of RNA phages, and hereby in particular the use of the VLP of RNA phage Qβ coat protein allows to achieve very high epitope density. The preparation of compositions of VLPs of RNA phage coat proteins with a high epitope density can be effected by using the teaching of this application.

The second attachment site, as defined herein, may be either naturally or non-naturally present with the antigen or the antigenic determinant. In the case of the absence of a suitable natural occurring second attachment site on the antigen or antigenic determinant, such a, then non-natural second attachment has to be engineered to the antigen.

As described above, four lysine residues are exposed on the surface of the VLP of Qβ coat protein. Typically these residues are derivatized upon reaction with a cross-linker molecule. In the instance where not all of the exposed lysine residues can be coupled to an antigen, the lysine residues which have reacted with the cross-linker are left with a crosslinker molecule attached to the ε-amino group after the derivatization step. This leads to disappearance of one or several positive charges, which may be detrimental to the solubility and stability of the VLP. By replacing some of the lysine residues with arginines, as in the disclosed Qβ coat protein mutants described below, we prevent the excessive disappearance of positive charges since the arginine residues do not react with the cross-linker. Moreover, replacement of lysine residues by arginines may lead to more defined antigen arrays, as fewer sites are available for reaction to the antigen.

Accordingly, exposed lysine residues were replaced by arginines in the following Qβ coat protein mutants and mutant Qβ3 VLPs disclosed in this application: Qβ-240 (Lys13-Arg; SEQ ID NO:6), Qβ-250 (Lys 2-Arg, Lys13-Arg; SEQ ID NO:8) and Qβ-259 (Lys 2-Arg, Lys16-Arg; SEQ ID NO:10). The constructs were cloned, the proteins expressed, the VLPs purified and used for coupling to peptide and protein antigens, as described in WO 02/056905. Qβ-251 (SEQ ID NO:9) was also constructed, and guidance on how to express, purify and couple the VLP of Qβ-251 coat protein can be found in WO 02/056905.

In a further embodiment, we disclose a Qβ mutant coat protein with one additional lysine residue, suitable for obtaining even higher density arrays of antigens. This mutant Qβ coat protein, Qβ-243 (Asn 10-Lys; SEQ ID NO:7), was cloned, the protein expressed, and the capsid or VLP isolated and purified as described in WO 02/056905, showing that introduction of the additional lysine residue is compatible with self-assembly of the subunits to a capsid or VLP. Thus, antigen or antigenic determinant arrays and conjugates, respectively, may be prepared using VLP of Qβ coat protein mutants. Further particularly favored methods of attachment of antigens to VLPs, and in particular to VLPs of RNA phage coat proteins are disclosed in WO 02/056905, the disclosures of which is herewith incorporated by reference in its entirety.

Epitope density on the VLP of RNA phage coat proteins can be modulated by the choice of cross-linker and other reaction conditions. For example, the cross-linkers Sulfo-GMBS and SMPH typically allow reaching high epitope density. Derivatization is positively influenced by high concentration of reactants, and manipulation of the reaction conditions can be used to control the number of antigens coupled to VLPs of RNA phage coat proteins, and in particular to VLPs of Qβ coat protein.

The selection and/or design of a non-natural second attachment site and preferred embodiments of second attachment sites are also disclosed in WO 02/056905.

In the most preferred embodiments, the at least one antigen or antigenic determinant comprises a single second attachment site or a single reactive attachment site capable of association with the first attachment sites on the core particle and the VLPs or VLP subunits, respectively. This ensures a defined and uniform binding and association, respectively, of the at least one, but typically more than one, preferably more than 10, 20, 40, 80, 120 antigens to the core particle and VLP, respectively. The provision of a single second attachment site or a single reactive attachment site on the antigen, thus, ensures a single and uniform type of binding and association, respectively leading to a very highly ordered and repetitive array. For example, if the binding and association, respectively, is effected by way of a lysine—(as the first attachment site) and cysteine—(as a second attachment site) interaction, it is ensured, in accordance with this preferred embodiment of the invention, that only one cysteine residue per antigen, independent whether this cysteine residue is naturally or non-naturally present on the antigen, is capable of binding and associating, respectively, with the VLP and the first attachment site of the core particle, respectively.

In a further preferred embodiment of the method of the invention, the at least one antigen or antigenic determinant is fused to the core particle and the virus-like particle, respectively. As outlined above, a VLP is typically composed of at least one subunit assembling into a VLP. Thus, in again a further preferred embodiment of the invention, the at least one antigen or antigenic determinant is fused to at least one subunit of the virus-like particle or of a protein capable of being incorporated into a VLP generating a chimeric VLP-subunit-antigen protein fusion.

Fusion of the antigen or antigenic determinant can be effected by insertion into the VLP subunit sequence, or by fusion to either the N- or C-terminus of the VLP-subunit or protein capable of being incorporated into a VLP. Hereinafter, when referring to fusion proteins of a peptide to a VLP subunit, the fusion to either ends of the subunit sequence or internal insertion of the peptide within the subunit sequence are encompassed.

Fusion may also be effected by inserting the antigen or antigenic determinant sequences into a variant of a VLP subunit where part of the subunit sequence has been deleted, that are further referred to as truncation mutants. Truncation mutants may have N- or C-terminal, or internal deletions of part of the sequence of the VLP subunit. For example, the specific VLP HBcAg with, for example, deletion of amino acid residues 79 to 81 is a truncation mutant with an internal deletion. Fusion of antigens to either the N- or C-terminus of the truncation mutants VLP-subunits also lead to embodiments of the invention. Likewise, fusion of an epitope into the sequence of the VLP subunit may also be effected by substitution, where for example for the specific VLP HBcAg, amino acids 79-81 are replaced with a foreign epitope. Thus, fusion, as referred to hereinafter, may be effected by insertion of the antigen sequence in the sequence of a VLP subunit, by substitution of part of the sequence of the VLP subunit with the antigen sequence, or by a combination of deletion, substitution or insertions.

The chimeric antigen-VLP subunit will be in general capable of self-assembly into a VLP. VLP displaying epitopes fused to their subunits are also herein referred to as chimeric VLPs. As indicated, the virus-like particle comprises or alternatively is composed of at least one VLP subunit. In a further embodiment of the invention, the virus-like particle comprises or alternatively is composed of a mixture of chimeric VLP subunits and non-chimeric VLP subunits, i.e. VLP subunits not having an antigen fused thereto, leading to so called mosaic particles. This may be advantageous to ensure formation of and assembly to a VLP. In those embodiments, the proportion of chimeric VLP-subunits may be 1, 2, 5, 10, 20, 30, 40, 50, 60, 70, 80, 90, 95% or higher.

Flanking amino acid residues may be added to either end of the sequence of the peptide or epitope to be fused to either end of the sequence of the subunit of a VLP, or for internal insertion of such peptidic sequence into the sequence of the subunit of a VLP. Glycine and serine residues are particularly favored amino acids to be used in the flanking sequences added to the antigen to be fused. Glycine residues confer additional flexibility, which may diminish the potentially destabilizing effect of fusing a foreign sequence into the sequence of a VLP subunit.

In a specific embodiment of the invention, the VLP is a Hepatitis B core antigen VLP. Fusion proteins to either the N-terminus of a HBcAg (Neyrinck, S. et al., Nature Med. 5:1157-1163 (1999)) or insertions in the so called major immunodominant region (MR) have been described (Pumpens, P. and Grens, E., Intervirology 44:98-114 (2001)), WO 01/98333), and are preferred embodiments of the invention. Naturally occurring variants of HBcAg with deletions in the MIR have also been described (Pumpens, P. and Grens, E., Intervirology 44:98-114 (2001), which is expressly incorporated by reference in their entirety), and fusions to the N- or C-terminus, as well as insertions at the position of the MIR corresponding to the site of deletion as compared to a wt HBcAg are further embodiments of the invention. Fusions to the C-terminus have also been described Pumpens, P. and Grens, E., Intervirology 44:98-114 (2001)). One skilled in the art will easily find guidance on how to construct fusion proteins using classical molecular biology techniques (Sambrook, J. et al., eds., Molecular Cloning, A Laboratory Manual, 2nd. edition, Cold Spring Harbor Laboratory Press, Cold Spring Harbor, N.Y. (1989), Ho et al., Gene 77:51 (1989)). Vectors and plasmids encoding HBcAg and HBcAg fusion proteins and useful for, the expression of a HBcAg and HBcAg fusion proteins have been described (Pumpens, P. & Grens, E. Intervirology 44: 98-114 (2001), Neyrinck, S. et al., Nature Med. 5:1157-1163 (1999)) and can be used in the practice of the invention. The insertion of an epitope into the MIR of HBcAg, resulting in a chimeric self-assembling HbcAg, is explicitly disclosed in Example 2. An important factor for the optimization of the efficiency of self-assembly and of the display of the epitope to be inserted in the MIR of HBcAg is the choice of the insertion site, as well as the number of amino acids to be deleted from the HBcAg sequence within the MIR (Pumpens, P. and Grens, E., Intervirology 44:98-114 (2001); EP 421,635; U.S. Pat. No. 6,231,864) upon insertion, or in other words, which amino acids form HBcAg are to be substituted with the new epitope. For example, substitution of HBcAg amino acids 76-80, 79-81, 79-80, 75-85 or 80-81 with foreign epitopes has been described (Pumpens, P. and Grens, E., Intervirology 44:98-114 (2001); EP0421635; U.S. Pat. No. 6,231,864). HBcAg contains a long arginine tail (Pumpens, P. and Grens, E., Intervirology 44:98-114 (2001)) which is dispensable for capsid assembly and capable of binding nucleic acids (Pumpens, P. and Grens, E., Intervirology 44:98-114 (2001)). HBcAg either comprising or lacking this arginine tail are both embodiments of the invention.

In a further preferred embodiment of the invention, the VLP is a VLP of a RNA phage. The major coat proteins of RNA phages spontaneously assemble into VLPs upon expression in bacteria, and in particular in E. coli. Specific examples of bacteriophage coat proteins which can be used to prepare compositions of the invention include the coat proteins of RNA bacteriophages such as bacteriophage Qβ (SEQ ID NO: 1; PIR Database, Accession No. VCBPQβreferring to Qβ CP and SEQ ID NO: 2; Accession No. AAA16663 referring to Qβ A1 protein), bacteriophage fr (SEQ ID NO:3; PIR Accession No. VCBPFR), and bacteriophage AP205 (SEQ ID NO: 18).

In a more preferred embodiment, the at least one antigen is fused to a Qβ coat protein. Fusion protein constructs wherein epitopes have been fused to the C-terminus of a truncated form of the A1 protein of Qβ, or inserted within the A1 protein have been described (Kozlovska, T. M., et al., Intervirology, 39:9-15 (1996)). The A1 protein is generated by suppression at the UGA stop codon and has a length of 329 aa, or 328 aa, if the cleavage of the N-terminal methionine is taken into account. Cleavage of the N-terminal methionine before an alanine (the second amino acid encoded by the Qβ CP gene) usually takes place in E. coli, and such is the case for N-termini of the Qβ coat proteins CP. The part of the A1 gene, 3′ of the UGA amber codon encodes the CP extension, which has a length of 195 amino acids. Insertion of the at least one antigen between position 72 and 73 of the CP extension leads to further embodiments of the invention (Kozlovska, T. M., et al., Intervirology 39:9-15 (1996)). Fusion of a antigen at the C-terminus of a C-terminally truncated Qβ A1 protein leads to further preferred embodiments of the invention. For example, Kozlovska et al., (Intervirology, 39: 9-15 (1996)) describe Qβ A1 protein fusions where the epitope is fused at the C-terminus of the Qβ CP extension truncated at position 19.

As described by Kozlovska et al. (Intervirology, 39: 9-15 (1996)), assembly of the particles displaying the fused epitopes typically requires the presence of both the A1 protein-antigen fusion and the wt CP to form a mosaic particle. However, embodiments comprising virus-like particles, and hereby in particular the VLPs of the RNA phage Qβcoat protein, which are exclusively composed of VLP subunits having at least one antigen fused thereto, are also within the scope of the present invention.

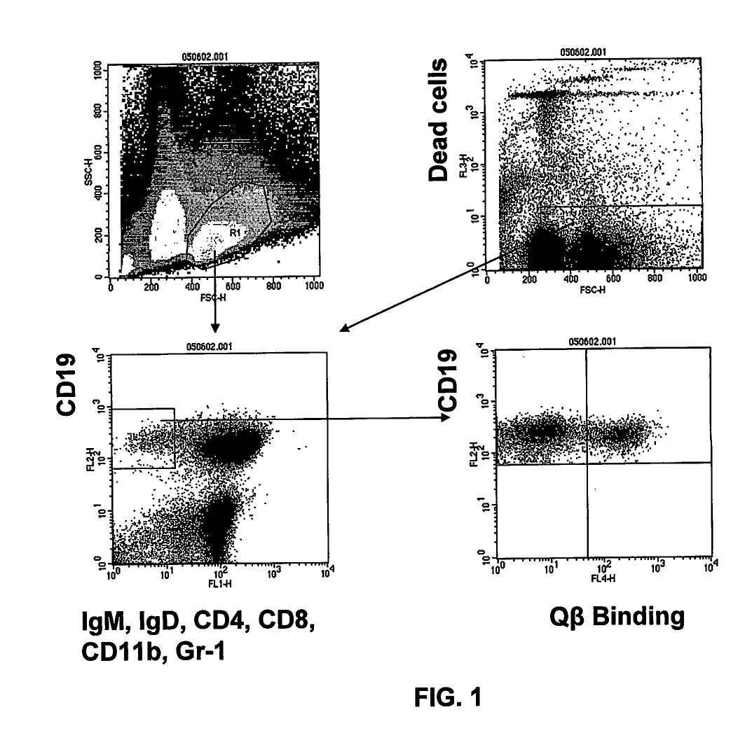

The production of mosaic particles may be effected in a number of ways. Kozlovska et al., Intervirolog, 39:9-15 (1996), describe two methods, which both can be used in the practice of the invention. In the first approach, efficient display of the fused epitope on the VLPs is mediated by the expression of the plasmid encoding the Qβ A1 protein fusion having a UGA stop codong between CP and CP extension in a E. coli strain harboring a plasmid encoding a cloned UGA suppressor tRNA which leads to translation of the UGA codon into Trp (pISM3001 plasmid (Smiley B. K., et al., Gene 134:3340 (1993))). In another approach, the CP gene stop codon is modified into UAA, and a second plasmid expressing the A1 protein-antigen fusion is cotransformed. The second plasmid encodes a different antibiotic resistance and the origin of replication is compatible with the first plasmid (Kozlovska, T. M., et al., Intervirology 39:9-15 (1996)). In a third approach, CP and the A1 protein-antigen fusion are encoded in a bicistronic manner, operatively linked to a promoter such as the Trp promoter, as described in FIG. 1 of Kozlovska et al., Intervirology, 39:9-15 (1996).

Further preferred embodiments of fusion of the at least one antigen to the VLP of the fr CP, to the coat protein of RNA phage MS-2, to a capsid protein of papillomavirus, and to a Ty protein capable of being incorporated into a Ty VLP, are disclosed in WO 02/056905, the disclosures of which is herewith incorporated by reference in its entirety.

Further VLPs suitable for fusion of antigens are, for example, Retrovirus-like-particles (WO9630523), HIV2 Gag (Kang, Y. C., et al, Biol. Chem. 380:353-364 (1999)), Cowpea Mosaic Virus (Taylor, K. M. et al., Biol. Chem. 380:387-392 (1999)), parvovirus VP2 VLP (Rueda, P. et al., Virology 263:89-99 (1999)), HBsAg (U.S. Pat. No. 4,722,840, EP0201416B1).

Examples of chimeric VLPs suitable for the practice of the invention are also those described in Intervirology 39:1 (1996). Further examples of VLPs contemplated for use in the invention are: HPV-1, HPV-6, HPV-11, HPV-16, HPV-18, HPV-33, HPV-45, CRPV, COPV, HIV GAG, Tobacco Mosaic Virus. Virus-like particles of SV-40, Polyomavirus, Adenovirus, Herpes Simplex Virus, Rotavirus and Norwalk virus have also been made, and chimeric VLPs of those VLPs are also within the scope of the present invention.

The antigen or antigenic determinant of the first and second composition used in the method of the invention may be any antigen or antigenic determinant of known or yet unknown provenance. In a preferred embodiment, the antigen or antigenic determinant is a recombinant antigen or a synthetic peptide. In another embodiment, the antigen or antigenic determinant is isolated from a natural source. It may be selected from the group consisting of without limitation polypeptides, carbohydrates, steroid hormones, organic molecules, bacteria, viruses, parasites, prions, tumors, self-molecules, non-peptide hapten molecules, allergens, or any other pathogen, or any other molecular compound, including without limitation inorganic molecules, against which it is desirable to have monoclonal antibodies, or it can be a recombinant antigen obtained from expression of suitable nucleic acid coding therefore. The selection of the antigen is, of course, dependent upon the antigen-specific B cell desired to select and the antibody desired that is expressed by the selected antigen-specific B cell of the invention. In particular, antibodies are desired that can be used in methods of treatment for allergies, cancer, drug abuse or other diseases or conditions associated with self antigens.

The selection of antigens or antigenic determinants for compositions used in the methods of the invention for the selection of B cells expressing antibodies useful in methods of treatment for allergies would be known to those skilled in the medical arts treating such disorders. Representative examples of such antigens or antigenic determinants include the following: bee venom phospholipase A2, Bet v I (birch pollen allergen), 5 Dol m V (white-faced hornet venom allergen), and Der p I (House dust mite allergen), as well as fragments of each which can be used to elicit immunological responses.

The selection of antigens or antigenic determinants for compositions used in the methods of the invention for the selection of B cells expressing antibodies useful in methods of treatment for cancer would be known to those skilled in the medical arts treating such disorders (see Renkvist et al., Cancer. Immunol. Immunother. 50:3-15 (2001) which is incorporated by reference), and such antigens or antigenic determinants are included within the scope of the present invention. Representative examples of such types of antigens or antigenic determinants, described in WO 02/056905, include without limitation the following: Her2 (breast cancer); GD2 (neuroblastoma); EGF-R (malignant glioblastoma); CEA (medullary thyroid cancer); CD52 (leukemia); human melanoma protein gp100; human melanoma protein gp100 epitopes such as amino acids 154-162 (sequence: KTWGQYWQV) (SEQ ID NO: 42), 209-217 (ITDQVPFSV) (SEQ ID NO: 43), 280-288 (YLEPGPVTA) (SEQ ID NO: 44), 457-466 (LLDGTATLRL) (SEQ ID NO: 45) and 476-485 (VLYRYGSFSV) (SEQ ID NO: 46); human melanoma protein melan-A/MART-1; human melanoma protein melan-A/MART-1 epitopes such as amino acids 27-35 (AAGIGILTV) (SEQ ID NO: 47) and 32-40 (ILTVILGVL) (SEQ ID NO:

48); tyrosinase and tyrosinase related proteins (e.g., TRP-1 and TRP-2); tyrosinase epitopes such as amino acids 1-9 (MLLAVLYCL) (SEQ ID NO: 49) and 369-377 (YMDGTMSQV) (SEQ ID NO: 50); NA17-A nt protein; NA17-A nt protein epitopes such as amino acids 38-64 (VLPDVFIRC) (SEQ ID NO: 51); MAGE-3 protein; MAGE-3 protein epitopes such as amino acids 271-279 (FLWGPRALV) (SEQ ID NO: 52); other human tumors antigens, e.g. CEA epitopes such as amino acids 571-579 (YLSGANLNL) (SEQ ID NO: 53); p53 protein; p53 protein epitopes such as amino acids 65-73 (RMPEAAPPV) (SEQ ID NO: 54), 149-157 (STPPPGTRV) (SEQ ID NO: 55) and 264-272 (LLGRNSFEV) (SEQ ID NO: 56); Her2/neu epitopes such as amino acids 369-377 (KIFGSLAFL) (SEQ ID NO: 57) and 654-662 (IISAVVGIL) (SEQ ID NO: 58); NYESO-1 peptides 157-165 and 157-167, 159-167; HPV16 E7 protein; HPV16 E7 protein epitopes such as amino acids 86-93 (TLGIVCPI) (SEQ ID NO: 59); as well as fragments or variants of each which can be used to elicit immunological responses.

The selection of antigens or antigenic determinants for compositions used in the methods of the invention for the selection of B cells expressing antibodies useful in methods of treatment for drug addiction, in particular recreational drug addiction, would be known to those skilled in the medical arts treating such disorders. Representative examples of such antigens or antigenic determinants include, for example, opioids and morphine derivatives such as codeine, fentanyl, heroin, morphium and opium; stimulants such as amphetamine, cocaine, MDMA (methylenedioxymethamphetamine), methamphetamine, methylphenidate and nicotine; hallucinogens such as LSD, mescaline and psilocybin; as well as cannabinoids such as hashish and marijuana.

The selection of antigens or antigenic determinants for compositions used in the methods of the invention for the selection of B cells expressing antibodies useful in methods of treatment for other diseases or conditions associated with self antigens would be also known to those skilled in the medical arts treating such disorders. Representative examples of such antigens or antigenic determinants are, for example, lymphotoxins (e.g. Lymphotoxin α (LT α), Lymphotoxin β (LT β)), and lymphotoxin receptors, Receptor activator of nuclear factor kappaB ligand (RANKL), vascular endothelial growth factor (VEGF) and vascular endothelial growth factor receptor (VEGF-R), Interleukin 17 and amyloid beta peptide (Aβ1-42), TNFα, MIF, MCP-1, SDF-1, Rank-L, M-CSF, Angiotensin II, Endoglin, Eotaxin, Grehlin, BLC, CCL21, IL-13, IL-17, IL-5, IL-8, IL-15, Bradykinin, Resistin, LHRH, GHRH, GIF, CRH, TRH and Gastrin, as well as fragments of each which can be used to elicit immunological responses.

In a particular embodiment of the invention, the antigen or antigenic determinant is selected from the group consisting of: (a) a recombinant polypeptide of HIV; (b) a recombinant polypeptide of Influenza virus (e.g., an Influenza virus M2 polypeptide or a fragment thereof); (c) a recombinant polypeptide of Hepatitis C virus; (d) a recombinant polypeptide of Hepatitis B virus (e) a recombinant polypeptide of Toxoplasma; (f) a recombinant polypeptide of Plasmodium falciparum; (g) a recombinant polypeptide of Plasmodium vivax; (h) a recombinant polypeptide of Plasmodium ovate; (i) a recombinant polypeptide of Plasmodium malariae; (j) a recombinant polypeptide of breast cancer cells; (k) a recombinant polypeptide of kidney cancer cells; (l) a recombinant polypeptide of prostate cancer cells; (m) a recombinant polypeptide of skin cancer cells; (n) a recombinant polypeptide of brain cancer cells; (o) a recombinant polypeptide of leukemia cells; (p) a recombinant profiling; (q) a recombinant polypeptide of bee sting allergy; (r) a recombinant polypeptide of nut allergy; (s) a recombinant polypeptide of pollen; (t) a recombinant polypeptide of house-dust; (u) a recombinant polypeptide of cat or cat hair allergy; (v) a recombinant protein of food allergies; (w) a recombinant protein of asthma; (x) a recombinant protein of Chlamydia; and (y) a fragment of any of the proteins set out in (a) (x).

As will be clear to those skilled in the art, certain embodiments of the invention involve the use of recombinant nucleic acid technologies such as cloning, polymerase chain reaction, the purification of DNA and RNA, the expression of recombinant proteins in prokaryotic and eukaryotic cells, etc. Such methodologies are well known to those skilled in the art and may be conveniently found in published laboratory methods manuals (e.g., Sambrook, J. et al., eds., Molecular Cloning, A Laboratory Manual, 2nd. edition, Cold Spring Harbor Laboratory Press, Cold Spring Harbor, N.Y. (1989); Ausubel, F. et al., eds., Current Protocols in Molecular Biology, John H. Wiley & Sons, Inc. (1997)). Fundamental laboratory techniques for working with tissue culture cell lines (Celis, J., ed., Cell Biology, Academic Press, 2nd edition, (1998)) and antibody-based technologies (Harlow, E. and Lane, D., “Antibodies: A Laboratory Manual,” Cold Spring Harbor Laboratory, Cold Spring Harbor, N.Y. (1988); Deutscher, M. P., “Guide to Protein Purification,” Meth. Enzymol. 128, Academic Press San Diego (1990); Scopes, R. K., “Protein Purification Principles and Practice,” 3rd ed., Springer-Verlag, New York (1994)) are also adequately described in the literature, all of which are incorporated herein by reference.

Preferred antigens used in the present invention can be synthesized or recombinantly expressed and coupled to the virus-like particle, or fused to the virus-like particle using recombinant DNA techniques. Exemplary procedures describing the attachment of antigens to virus-like particles are disclosed in WO 00/32227, in WO 01/85208 and in WO 02/056905, the disclosures of which are herewith incorporated by reference in its entirety.

The following examples are illustrative only and are not intended to limit the scope of the invention as defined by the appended claims. It will be apparent to those skilled in the art that various modifications and variations can be made in the methods of the present invention without departing from the spirit and scope of the invention. Thus, it is intended that the present invention cover the modifications and variations of this invention provided they come within the scope of the appended claims and their equivalents.

All patents and publications referred to herein are expressly incorporated by reference in their entirety.

### EXAMPLES

Enzymes and reagents used in the experiments that follow included: T4 DNA ligase obtained from New England Biolabs; Taq DNA Polymerase, QIAprep Spin Plasmid Kit, QIAGEN Plasmid Midi Kit, QiaExH Gel Extraction Kit, QIAquick PCR Purification Kit obtained from QIAGEN; QuickPrep Micro mRNA Purification Kit obtained from Pharmacia; SuperScript One step RT PCR Kit, fetal calf serum (FCS), bacto tryptone and yeast extract obtained from Gibco BRL; Oligonucleotides obtained from Microsynth (Switzerland); restriction endonucleases obtained from Boehringer Mannheim, New England Biolabs or MBI Fermentas; Pwo polymerase and dNTPs obtained from Boehringer Mannheim. HP 1 medium was obtained from Cell culture technologies (Glattbrugg, Switzerland). All standard chemicals were obtained from Fluka Sigma Aldrich, and all cell culture materials were obtained from TPP. DNA manipulations were carried out using standard techniques. DNA was prepared according to manufacturer instruction either from a 2 ml bacterial culture using the QIAprep Spin Plasmid Kit or from a 50 ml culture using the QIAGEN Plasmid Midi Kit. For restriction enzyme digestion, DNA was incubated at least 2 hours with the appropriate restriction enzyme at a concentration of 5 10 units (U) enzyme per mg DNA under manufacturer recommended conditions (buffer and temperature). Digests with more than one enzyme were performed simultaneously if reaction conditions were appropriate for all enzymes, otherwise consecutively. DNA fragments isolated for further manipulations were separated by electrophoresis in a 0.7 to 1.5% agarose gel, excised from the gel and purified with the QiaExH Gel Extraction Kit according to the instructions provided by the manufacturer. For ligation of DNA fragments, 100 to 200 pg of purified vector DNA were incubated overnight with a threefold molar excess of the insert fragment at 16° C. in the presence of 1 U T4 DNA ligase in the buffer provided by the manufacturer (total volume: 10 20 μl). An aliquot (0.1 to 0.5 μl) of the ligation reaction was used for transformation of E. coli XL1 Blue (Stratagene).

Transformation was done by electroporation using a Gene Pulser (BioRAD) and 0.1 cm Gene Pulser Cuvettes (BioRAD) at 200 Ohm, 25 μF, 1.7 kV. After electroporation, the cells were incubated with shaking for 1 h in 1 ml S.O.B. medium (Miller, 1972) before plating on selective S.O.B. agar.

### Example 1

Construction of HBcAg1-185-Lys.

Hepatitis core Antigen (HBcAg) 1-185 (SEQ ID NO: 14) was modified as described in Example 24 of WO 02/056905. A part of the c/el epitope (residues 72 to 88) region (Proline 79 and Alanine 80) was genetically replaced by the peptide Gly-Gly-Lys-Gly-Gly (SEQ ID NO: 60) (resulting in HBcAg1-185-Lys construct, SEQ ID NO: 15). The introduced Lysine residue contains a reactive amino group in its side chain that can be used for intermolecular chemical crosslinking of HBcAg particles with any antigen containing a free cysteine group. PCR methods and conventional cloning techniques were used to prepare the HBcAg1-185-Lys gene.

The Gly-Gly-Lys-Gly-Gly sequence was inserted by amplifying two separate fragments of the HBcAg gene from pEco63, as described in Example 24 of WO 02/056905, and subsequently fusing the two fragments by PCR to assemble the full length gene. The following PCR primer combinations were used:

fragment 1:

| | | |
|---|---|---|
| Primer 1: | EcoRIHBcAg(s) | (SEQ ID NO: 33) |
| Primer 2: | Lys-HBcAg(as) | (SEQ ID NO: 34) |

fragment 2:

| | | |
|---|---|---|
| Primer 3: | Lys-HBcAg(s) | (SEQ ID NO: 35) |
| Primer 4: | HBcAgwtHindIIII | |

(SEQ ID NO: 36)

CGCGTCCCAAGCTTCTAACATTGAGATTCCCGAGATTG

| | | |
|---|---|---|
| Assembly: | | |
| Primer 1: | EcoRIHBcAg(s) | (SEQ ID NO: 33) |
| Primer 2: | HBcAgwtHindIIII | |

The assembled full length gene was then digested with the EcoRI (GAATTC) and HindIII (AAGCTT) enzymes and cloned into the pKK vector (Pharmacia) cut at the same restriction sites.

### Example 2

Fusion of a peptide epitope in the MIR region of HbcAg.

The residues 79 and 80 of HBcAg1-185 were substituted with the epitope CεH3 of sequence VNLTWSRASG (SEQ ID NO: 37). The CεH3 sequence stems from the sequence of the third constant domain of the heavy chain of human IgE. The epitope was inserted in the HBcAg1-185 sequence using an assembly PCR method. In the first PCR step, the HBcAg1-185 gene originating from ATCC clone pEco63 and amplified with primers HBcAg-wt EcoRI fwd and HBcAg-wt Hind III rev was used as template in two separate reactions to amplify two fragments containing sequence elements coding for the C□H3 sequence. These two fragments were then assembled in a second PCR step, in an assembly PCR reaction.

Primer combinations in the first PCR step: CεH3fwd with HBcAg-wt Hind III rev, and HBcAg-wt EcoRI fwd with CεH3rev. In the assembly PCR reaction, the two fragments isolated in the first PCR step were first assembled during 3 PCR cycles without outer primers, which were added afterwards to the reaction mixture for the next 25 cycles. Outer primers: HBcAg-wt EcoRI fwd and HBcAg-wt Hind III rev.

The PCR product was cloned in the pKK223.3 using the EcoRI and Hind III sites, for expression in E. coli (see Example 23 of WO 02/056905). The chimeric VLP was expressed in E. coli and purified as described in Example 23 of WO 02/056905. The elution volume at which the HBcAg1-185-CεH3 eluted from the gel filtration showed assembly of the fusion proteins to a chimeric VLP.

Primer sequences:
CεH3fwd:

(SEQ ID NO: 38)

5′GTT AAC TTG ACC TGG TCT CGT GCT TCT GGT GCA TCC
AGG GAT CTA GTA GTC 3′;

(SEQ ID NO: 39)

```
      V N L T W S R A S G A S
  R D L V V
CεH3rev:
```

(SEQ ID NO: 40)

5′ACC AGA AGC ACG AGA CCA GGT CAA GTT AAC ATC TTC
CAA ATT ATT ACC CAC 3′

(SEQ ID NO: 41)

```
                                                                                      D E
  L N N G V
HBcAg-wt EcoRI fwd:
```

(SEQ ID NO: 33)

5′CCGgaattcATGGACATTGACCCTTATAAAG
HBcAg-wt Hind III rev:

(SEQ ID NO: 36)

5′CGCGTCCCaagcttCTAACATTGAGATTCCCGAGATTG

In the following Examples (Example 3 to Example 7) further two preferred embodiments of the first and/or second core particle of the first and/or second composition have been used, i.e., first, a strategically modified Hepatitis core Antigen (HBcAg), typically referred throughout this specification as HBcAg-lys-2cys-Mut (SEQ ID NO: 62), and, second, a virus-like particle of the RNA-phage Qb.

The modification of the strategically modified Hepatitis core Antigen (HBcAg) comprises, first, the introduction of a lysine residue within its c/el epitope (being residues 72 to 88 of SEQ ID NO: 14), which is located in the tip region on the surface of the Hepatitis B virus capsid (HBcAg). A part of this region (Proline 79 and Alanine 80) was genetically replaced by the peptide Gly-Gly-Lys-Gly-Gly (SEQ ID NO: 60) leading to the “HBcAg-Lys construct” (SEQ ID NO: 15). The introduced Lysine residue contains a reactive amino group in its side chain that can be used for intermolecular chemical crosslinking of HBcAg particles with any antigen containing a free cysteine group as described herein. Second, cysteine residues at positions corresponding to 48 and 107 in SEQ ID NO:14 have been replaced by serine residues resulting in the HBcAg-lys-2cys-Mut (SEQ ID NO: 62). The experimental setup for the production of the HBcAg-lys-2cys-Mut is described in Examples 23, 24 and 31 of WO 02/056905.

The other preferred embodiment of the first and/or second core particle used within the examples is the virus-like particle, which comprises, or alternatively consists essentially of, or alternatively consists of recombinant proteins, or fragments thereof, of the RNA-bacteriophage Qβ, wherein the recombinant proteins comprise, or alternatively consist essentially of, or alternatively consist of a mixture of coat proteins having amino acid sequences of SEQ ID NO: 1 and of SEQ ID NO: 2. Typically and preferably, the indicated RNA-phage Qβwas expressed in E. coli using the expression vector, pQβ10, and purified as described in Cielens, I., et al. (2000) FEBS Lett 482: 261-264.

For the sake of simplicity the HBcAg-lys-2cys-Mut as well as the RNA-bacteriophage Qβare referred within the following examples as HBcAg and Qβ.

### Example 3

Staining of specific B cells in mice immunized with Qβ, AP205 or HBcAg containing a reactive Lys in the immunodominant region

Mice were immunized intravenously with 10 μg Qβ, AP205 or HBcAg diluted in PBS and spleens were removed 21 days after immunization.

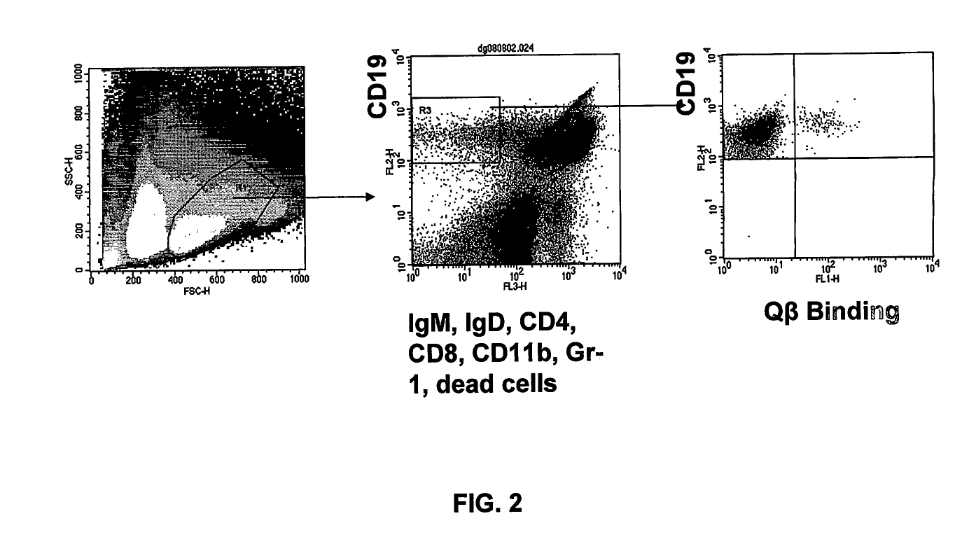

For detection of B cells expressing Qb-, AP205- or HbcAg-specific surface Ig, single cells suspensions of splenocytes were incubated with Qb, AP205 or HBcAg capsids (1 μg/ml) followed by a polyclonal rabbit anti-Qb, anti-AP205 or anti-HBcAg antiserum and Cy5-conjugated donkey anti-rabbit IgG serum (Jackson ImmunoResearch Laboratories, West Grove, Pa.). Cells were stained with a mixture of FITC-conjugated antibodies (anti-IgD, clone 11-26; goat anti-IgM serum, Jacksommuno Research Laboratories; anti CD4, clone GK1.5; anti CD8, clone 53-6.7; anti-CD11b, clone M1/70; anti-Gr-1, clone RB6-8C5) and PE-conjugated anti-CD19 (clone 1D3) to detect isotype-switched B cells (FIG. 1). Alternatively, Qb particles were labelled with the fluorochrome Alexa 488, using the Alexa Fluor 488 Protein Labeling Kit (Molecular Probes) according to the manufacturer's instructions. To detect isotype-switched B cells, single cells suspensions of splenocytes were then incubated with the labeled Qb, stained with biotinylated anti-IgD (eBioscience) and anti-IgM antibodies (Jackson ImmunoResearch) followed by streptavidin-Cy-Chrome, a mixture of Cy-Chrome-conjugated antibodies (anti-CD4, anti-CD8, anti-CD11b) and PE-conjugated anti-CD 19 (FIG. 2).

After staining, cells were resuspended in 0.5 mg/ml propidium iodide for exclusion of dead cells. Staining was performed at 4° C. for 30 min in PBS containing 2% FCS and 0.05% NaN3; Fc-receptors were blocked with anti-mouse CD16/32 (clone 2.4G2). Antibodies were purchased from BD Biosciences unless otherwise noted.

### Example 4

Coupling of peptide D2 to Qβ Capsid and Hepatitis B Core

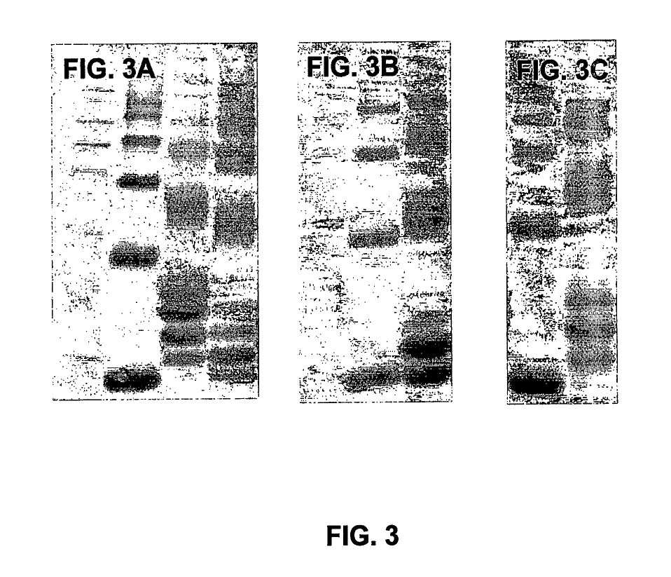

A solution of 125 μM Q capsid or hepatitis B core (HBcAg(1-149) with Lysin in tip) (in 20 mM Hepes, 150 mM NaCl pH 7.2) was reacted for 45 minutes with a 50-fold molar excess of SMPH (Pierce), diluted from a stock solution in DMSO, at 25° C. on a rocking shaker. The reaction solution was subsequently dialyzed twice for 2 hours against 1 L of 20 mM Hepes, 150 mM NaCl, pH 7.2 at 4° C. The dialyzed VLP-reaction mixture was then reacted with the D2 peptide from Salmonella Typhi for four hours at 25° C. on a rocking shaker (end concentrations: 125 μM VLP, 1.25-2.5 mM D2 for high coupling density or 625 μM D2 for low coupling density). The reaction was dialyzed against 1L 20 mM Hepes, 150 mM NaCl, pH 7.2 at 4° C. (2×2 hours). Coupling products were analysed by SDS-PAGE (FIG. 3).

### Example 5

Staining of Antigen-Specific B Cells using Qβ Containing an Antigen Coupled to a Reactive Lys in the Immunodominant Region

Mice were immunized intravenously with 10 μg peptide D2 coupled to HBcAg (diluted in PBS) and spleens were removed 21 days after immunization.

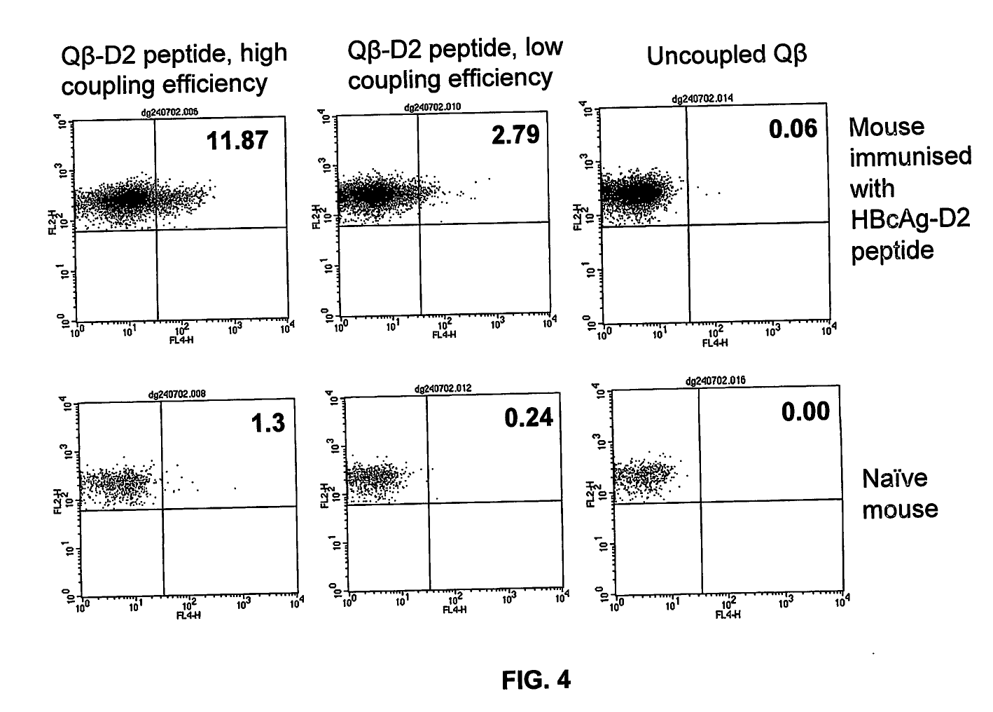

For detection of B cells specific for the D2 peptide (Salmonella Typhi) (FIG. 4), single cells suspensions of splenocytes were incubated with D2 coupled to Qb capsids (1 μg/ml) followed by a polyclonal rabbit anti-Qb antiserum and Cy5-conjugated donkey anti-rabbit IgG serum (Jackson ImmunoResearch Laboratories, West Grove, Pa.). Cells were stained with a mixure of FITC-conjugated antibodies (anti-IgD, clone 11-26; goat anti-IgM serum, JacksonImmuno Research Laboratories; anti CD4, clone GK1.5; anti CD8, clone 53-6.7; anti-CD11b, clone M1/70; anti-Gr-1, clone RB6-8C5) and PE-conjugated anti-CD19 (clone 1D3) to detect isotype-switched B cells. After staining cells were resuspended in 0.5 mg/ml propidium iodide for exclusion of dead cells. Staining was performed at 4° C. for 30 min in PBS containing 2% FCS and 0.05% NaN3; Fc-receptors were blocked with anti-mouse CD16/32 (clone 2.4G2). Antibodies were purchased from BD Biosciences unless otherwise noted.

### Example 6

Staining of High Affinity B Cells using Qβ Exhibiting Few Antigens Coupled to it.

Mice were immunized intravenously with 10 μg peptide D2 coupled to HBcAg (diluted in PBS) and spleens were removed 21 days after immunization. For detection of high affinity B cells specific for the D2 peptide (Salmonella Typhi) (FIG. 4, upper middle panel), single cells suspensions of splenocytes were incubated with D2 coupled to Qβ with low efficiency (Qβ-D2 concentration 1 μg/ml) followed by a polyclonal rabbit anti-Qb antiserum and Cy5-conjugated donkey anti-rabbit IgG serum (Jackson ImmunoResearch Laboratories, West Grove, Pa.). Cells were stained with a mixture of FITC-conjugated antibodies (anti-IgD, clone 11-26; goat anti-IgM serum, JacksonImmuno Research Laboratories; anti CD4, clone GK1.5; anti CD8, clone 53-6.7; anti-CD11b, clone M1/70; anti-Gr-1, clone RB6-8C5) and PE-conjugated anti-CD19 (clone 1D3) to detect isotype-switched B cells. After staining cells were resuspended in 0.5 mg/ml propidium iodide for exclusion of dead cells. Staining was performed at 4° C. for 30 min in PBS containing 2% FCS and 0.05% NaN3; Fc-receptors were blocked with anti-mouse CD16/32 (clone 2.4G2). Antibodies were purchased from BD Biosciences unless otherwise noted.

### Example 7

Single Cell Sorting of Specific B Cells upon Staining with Qβ

Single B cells specific for Qβ stained as in example 3, were sorted as described in “Current protocols in immunology, John Wiley & sons Inc., Coligan J. et. al.; Volume 1, Chapter 5 (5.0.1-5.8.23); 2002” using a FACSVantage (Becton Dickinson) into 96-well plates containing 6.105 rat thymocytes/well in 200 μl RPMI medium supplemented with 7.5% FCS, 1 ng/ml IL-6 (R&D Systems) and 25 μg/ml LPS (Sigma-Aldrich). Cells were grown 8 days at 37° C. and 5% CO₂. Specific antibody production was detected by ELISA, which was performed according to standard protocols. In brief, 10 mg Qb in coating buffer (0.1 M NaHCO, pH 9.6) was coated onto ELISA plates (Nunc Immuno Maxisorb) at 4° C. overnight. After a blocking step, supernatants from B cell cultures were added to the plates and incubated for 4 hours at 37° C. Specific antibody bound to the plate was detected using HRPO-conjugated anti IgG antibody (Sigma-Aldrich). Plates were developed with OPD substrate buffer (0.5 mg/ml OPD, 0.01% H₂O₂, 0.066 M Na2HPO4, 0.038 M citric acid, pH 5.0; 100 μl each well) and plates were read in an ELISA reader at 450 nm.

### Example 8

Cloning of VH (Variable Domain of the Heavy Chain) Segments from Qβ-Specific B Cells

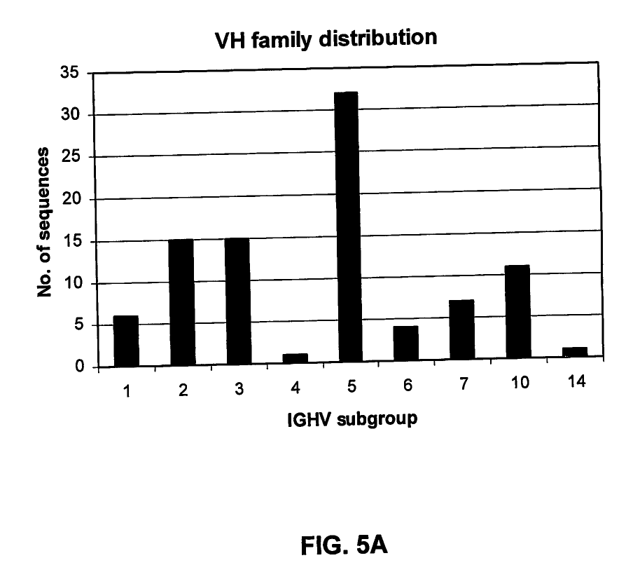

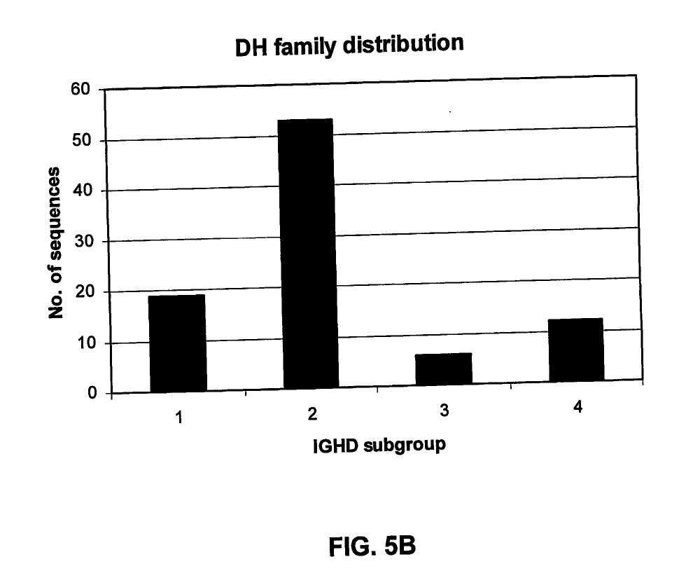

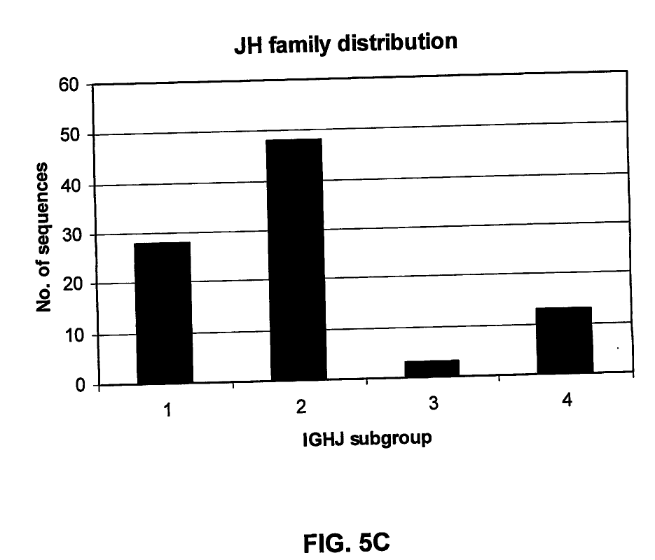

0.5-1×105 Qβ-specific B cells were sorted into TR1 Reagent (Molecular Research Center) with a FACSVantage (Becton Dickinson). Total RNA was extracted from sorted cells according to the manufacturer's instructions. First strand cDNA was synthesized from total RNA using random nonamer primers and SuperScript II reverse transcriptase (Invitrogen). For amplification of VH sequences primer sets incorporating all mouse VH sequences collected in the Kabat database were used (Table I, described in A. Krebber et al., Journal of Immunological Methods 201 (1997) 35-55). Advantage 2 Polymerase Mix (Clontech) was used for amplification. PCR reactions were performed in 50 μl volumes, containing 1 μl cDNA, 2 μM of primer HB and HF primer mixes, 200 μM dNTPs and 10× Advantage 2 PCR Buffer supplied by the manufacturer. The cycling conditions were 1 min at 95° C., followed by 30 cycles of 30 s at 95° C., 30 s at 58° C. and 1 min at 68° C. and a final elongation step of 1 min at 68° C. The full length PCR products were purified by preparative gel electrophoresis using the QIAquick gel extraction kit (Qiagen). Purified VH DNA (4 μl) was cloned into the pCR®II-TOPO® plasmid vector using the TOPO TA Cloning® kit (Invitrogen) according to the manufacturer's protocol and transformed into E. coli TOP10F′. Plasmid DNA of recombinant clones was purified from overnight cultures using the QIAprep spin miniprep kit (Qiagen) and DNA sequences were determined by cycle sequencing. Sequences were matched against a database of murine immunoglobulin germline sequences (IMGT) to determine the families of the V (variable segment, FIG. 5A), D (diversity segment, FIG. 5B), and J (joining segment, FIG. 5C) segments (see http://imgt.cines.fr/cgi-bin/IMGTdnap.jv?livret=0&Option=mouseIg, IMGT, the international ImMunoGeneTics databases as described in Lefranc, M.-P. et al., Nucleic Acids Res., 31, 370-310 (2003)).

TABLE I

Primers for the amplification of mouse Ig
heavy-chain variable domains
The sequences are given using the IUPAC nomen-
clature of mixed bases (R = A or G; Y = C or T;
M = A or C; K = G or T; S = C or G; W = A or T;
H = A or C or T; B = G or C or T; V = A or C or
G; D = A or G or T).

| Designation | | |
|---|---|---|
| Krebber et al.* | OLIGO SEQUENCE | SEQ ID NO: |
| HB1 | GAKGTRMAGCTTCAGGAGTC | 63 |
| HB2 | GAGGTBCAGCTBCAGCAGTC | 64 |
| HB3 | CAGGTGCAGCTGAAGSASTC | 65 |
| HB4 | GAGGTCCARCTGCAACARTC | 66 |
| HB5 | CAGGTYCAGCTBCAGCARTC | 67 |
| HB6 | CAGGTYCARCTGCAGCAGTC | 68 |
| HB7 | CAGGTCCACGTGAAGCAGTC | 69 |
| HB8 | GAGGTGAASSTGGTGGAATC | 70 |
| HB9 | GAVGTGAWGYTGGTGGAGTC | 71 |
| HB10 | GAGGTGCAGSKGGTGGAGTC | 72 |
| HB11 | GAKGTGCAMCTGGTGGAGTC | 73 |
| HB12 | GAGGTGAAGCTGATGGARTC | 74 |
| HB13 | GAGGTGCARCTTGTTGAGTC | 75 |
| HB14 | GARGTRAAGCTTCTCGAGTC | 76 |
| HB15 | GAAGTGAARSTTGAGGAGTC | 77 |
| HB16 | CAGGTTACTCTRAAAGWGTSTG | 78 |
| HB17 | CAGGTCCAACTVCAGCARCC | 79 |
| HB18 | GATGTGAACTTGGAAGTGTC | 80 |
| HB19 | GAGGTGAAGGTCATCGAGTC | 81 |
| HF1 | GAGGAAACGGTGACCGTGGT | 82 |
| HF2 | GAGGAGACTGTGAGAGTGGT | 83 |
| HF3 | GCAGAGACAGTGACCAGAGT | 84 |
| HF4 | GAGGAGACGGTGACTGAGGT | 85 |

*Journal of Immunological Methods 201 (1997), p. 35-55.

1. A method for selecting at least one antigen-specific B cell from a mixture of cells, said method comprising:
(A) providing a mixture of cells comprising B cells;
(B) providing a first composition comprising:
(a) a first core particle with at least one first attachment site; and
(b) at least one antigen or antigenic determinant with at least one second attachment site, wherein said second attachment site being selected from the group consisting of:
(i) an attachment site not naturally occurring with said antigen or antigenic determinant; and
(ii) an attachment site naturally occurring with said antigen or antigenic determinant;
wherein said second attachment site associates through at least one covalent bond with said first attachment site such that said antigen or antigenic determinant and said first core particle interact through said association to form an ordered and repetitive antigen array;
(C) contacting said mixture of cells with said first composition;
(D) labeling said first composition with a first labeling compound;
(E) labeling said B cells in said mixture of cells with a second labeling compound; and
(F) selecting at least one B cell which is positive for said first and said second labeling compound.

2. (canceled)

3. The method of claim 1, wherein said first core particle is a virus-like particle.

4. The method of claim 3, wherein said at least one antigen or antigenic determinant is bound to said virus-like particle.

5. (canceled)

6. (canceled)

7. The method of claim 3, wherein said virus-like particle comprises recombinant proteins, or fragments thereof, of an RNA-phage.

8. (canceled)

9. The method of claim 3, wherein said virus-like particle comprises recombinant proteins, or fragments thereof, of RNA-phage Qβ.

10-14. (canceled)

15. The method of claim 1, further comprising the step of isolating said at least one antigen-specific B cell which is positive for said first and said second labeling compound.

16-18. (canceled)

19. The method of claim 1, wherein said first labeling compound is a first fluorochrome.

20. The method of claim 1, wherein said second labeling compound is a second fluorochrome.

21-31. (canceled)

32. The method of claim 1, wherein said labeling of said B cells is effected with a first set of at least one first targeting molecule, wherein said first targeting molecule is specific for at least one B cell marker, and wherein said first targeting molecule is labeled with said second labeling compound, wherein said second labeling compound is a second fluorochrome.

33. (canceled)

34. (canceled)

35. The method of claim 32, wherein said first targeting molecule is F(ab′)2 specific for IgG.

36. (canceled)

37. The method of claim 1, further comprising labeling said mixture with a second set of at least one second additional targeting molecule, wherein said at least one second targeting molecule is specific for at least one marker unique for cells other than isotype-switched B cells, and wherein said at least one second targeting molecule is labeled with a third labeling compound.

38. (canceled)

39. The method of claim 37, wherein said first labeling compound is a first fluorochrome, said second labeling compound is a second fluorochrome, and said third labeling compound is a third fluorochrome, said first, second, and third fluorochromes yielding different colors upon activation.

40-43. (canceled)

44. The method of claim 1, wherein said mixture of cells is a mixture of splenocytes from immunized animals, said animals being immunized with a second composition comprising:
(a) a second core particle with at least one first attachment site; and
(b) at least one antigen or antigenic determinant with at least one second attachment site, wherein said second attachment site is selected from the group consisting of:
(i) an attachment site not naturally occurring with said antigen or antigenic determinant; and
(ii) an attachment site naturally occurring with said antigen or antigenic determinant,
wherein said second attachment site is associates with said first attachment site; and
wherein said antigen or antigenic determinant and said second core particle interact through said association to form an ordered and repetitive antigen array.

45. (canceled)

46. (canceled)

47. The method of claim 44, wherein said second core particle is a virus-like particle, wherein said at least one antigen or antigenic determinant is bound to said virus-like particle.

48. (canceled)

49. The method of claim 47, wherein said virus-like particle comprises proteins selected from the group consisting of:
(a) recombinant proteins of Hepatitis B virus;
(b) recombinant proteins of measles virus;
(c) recombinant proteins of Sindbis virus;
(d) recombinant proteins of Rotavirus;
(e) recombinant proteins of Foot-and-Mouth-Disease virus;
(f) recombinant proteins of Retrovirus;
(g) recombinant proteins of Norwalk virus;
(h) recombinant proteins of Alphavirus;
(i) recombinant proteins of human Papilloma virus;
(j) recombinant proteins of Polyoma virus;
(k) recombinant proteins of bacteriophages;
(l) recombinant proteins of RNA-phages;
(m) recombinant proteins of Ty;
(n) recombinant proteins of Qβ-phage;
(o) recombinant proteins of GA-phage;
(p) recombinant proteins of fr-phage;
(q) fragments of any of the recombinant proteins from (a) to (p); and
(r) variants of any of the recombinant proteins from (a) to (q).

50. (canceled)

51. (canceled)

52. The method of claim 47, wherein said virus-like particle is a virus like particle of an RNA-phage, wherein said RNA phase is selected from the group consisting of:
(a) bacteriophage Qβ;
(b) bacteriophage R17;
(c) bacteriophage fr;
(d) bacteriophage GA;
(e) bacteriophage SP;
(f) bacteriophage MS2;
(g) bacteriophage M11;
(h) bacteriophage MX1;
(i) bacteriophage NL95;
(j) bacteriophage f2;
(k) bacteriophage PP7; and
(l) bacteriophage AP205.

53. The method of claim 47, wherein said virus-like particle comprises recombinant proteins, or fragments thereof, of RNA-phage Qβ.

54-56. (canceled)

57. The method of claim 44, wherein said antigen or antigenic determinant of said second composition is the same as said antigen or antigenic determinant of said first composition.

58. (canceled)

59. A method for generating monoclonal antibodies comprising the steps of providing at least one antigen-specific B cell selected by the method any one of the methods of claim 1 or claim 44 and fusing said at least one antigen-specific B cell with a myeloma cell line.

60. (canceled)

61. A method for generating monoclonal antibodies or antibody fragments comprising the steps of isolating at least one genetic element encoding the immunoglobulin or parts of the immunoglobulin expressed by said at least one antigen-specific B cell selected by the method of claim 1 and expressing said genetic element.

62. The method of claim 61, wherein said genetic element is expressed as a fusion molecule.
